(19) United States
(12) Patent Application Publication
(10) Pub. No.: US 2009/0048273 A1
(43) Pub. Date: Feb. 19, 2009

(54) PREMATURE OVULATION PREVENTIVE AGENT

(75) Inventors: Shuichi Furuya, Tsukuba-shi (JP); Masami Kusaka, Tsukuba-shi (JP)

Correspondence Address: EDWARDS ANGELL PALMER & DODGE LLP, P.O. BOX 55874, BOSTON, MA, 02205, US

(73) Assignee: TAKEDA PHARMACEUTICAL COMPANY LIMITED, Osaka (JP)

(21) Appl. No.: 11/996,269

(22) PCT Filed: Jul. 21, 2006
(86) PCT No.: PCT/JP2006/314943

§ 371 Date: Jan. 18, 2008

(30) Foreign Application Priority Data
Jul. 22, 2005 (JP) 212973/2005

(51) Int. Cl. C07D 495/04; A61K 31/519; A61P 15/08

(52) U.S. Cl. 514/260.1; 544/278

(57) ABSTRACT

The present invention provides a premature ovulation inhibitor for use in in vitro fertilization or embryo transfer process, which contains a nonpeptidic compound having a gonadotropin releasing hormone antagonistic action. The premature ovulation inhibitor for use in in vitro fertilization or embryo transfer process of the present invention is low toxic, permits oral administration, and has a superior inhibitory effect on premature ovulation in in vitro fertilization or embryo transfer process.

### TECHNICAL FIELD

The present invention relates to a pharmaceutical use of a nonpeptidic compound having a gonadotropin releasing hormone antagonistic action, and specifically relates to a premature ovulation inhibitor for use in in vitro fertilization or embryo transfer process.

### BACKGROUND OF THE INVENTION

Secretion of anterior pituitary hormone is regulated by peripheral hormones secreted from respective hormone target organs and releasing or inhibiting hormone (hereinafter these hormone groups are generically referred to as hypothalamic hormones in the present specification) secreted from hypothalamus, which is the upper nerve of anterior pituitary. Heretofore, 9 kinds of hormones have been confirmed to be present as hypothalamic hormones, for example, thyrotropin releasing hormone (TRH), gonadotropin releasing hormone {GnRH: also referred to as luteinizing hormone releasing hormone (LH-RH)}, and the like. These hypothalamic hormones are assumed to express their hormone action and the like via receptors considered to be present in the anterior pituitary, and analyses of receptor genes, including those of human, specific to these hormones are ongoing. Accordingly, an antagonist or agonist specific and selective to those receptors will control action of hypothalamic hormones and regulate secretion of anterior pituitary hormones. Consequently, the antagonist or agonist is expected to prevent or treat diseases dependent on those anterior pituitary hormones.

WO00/56739 and WO04/067535 disclose that a gonadotropin releasing hormone (GnRH) antagonist is useful as an agent for the prophylaxis or treatment of sex hormone dependent cancers (e.g., prostate cancer, uterine cancer, breast cancer, pituitary tumor, etc.), prostatic hyperplasia, uterine fibroid, endometriosis, precocious puberty, amenorrhea, premenstrual syndrome, multilocular ovary syndrome, acne and the like; a reproduction regulator for males and females (e.g., birth control agent, menstrual cycle regulator, etc.); a contraceptive drug for males and females; an ovulation inducing agent for females; an infertility therapy agent; and as an estrus regulator for animals, a meat quality improver for edible meat, and a growth promoter for animals in the animal husbandry field.

For infertility treatment, use of in vitro fertilization is on the rise. It is used for infertile patients with fallopian tube disease, endometriosis, oligospermia, antisperm antibody, and other infertility of unknown cause.

In in vitro fertilization, a treatment for stimulating the ovary such as a combined use of clomiphene citrate and HMG (Human Menopausal Gonadotropin), single administration of HMG, a combined use of a GnRH agonist and HMG, and the like is performed to mature the oocyte. Generally, after suitable maturation of the ovarian follicle, an oocyte is transvaginally collected under ultrasound supervision, and the collected oocyte is subjected to in vitro fertilization. An incubated embryo is transferred into the uterine cavity and, when successful, implanted and results in pregnancy. For infertility treatment, in vitro fertilization where ovum collection to embryo transfer are performed in a sequence of procedures, as well as frozen embryo transfer, gamete intrafallopian transfer and the like may be performed.

Use of a peptidic GnRH antagonist that lowers the LH (Luteinizing hormone) level in in vitro fertilization (IVF) has been reported (European Journal of Obstetrics & Gynecology and Reproductive Biology 115S (2004) S44-S56).

However, peptidic compounds are still problematic in many respects such as oral absorbability, administration form, dose, stability of pharmaceutical agent, sustainability of action, metabolic stability, and the like. There is a strong demand for a nonpeptidic compound having a gonadotropin releasing hormone antagonistic action, which is superior in oral absorbability, can enhance or assist in vitro fertilization, and does not cause a transient pituitary gonadotropin action (acute action).

An object of the present invention is to provide a premature ovulation inhibitor for use in in vitro fertilization or embryo transfer process, which has superior GnRH antagonistic action, is low toxic, superior in oral absorbability, sustainability of action, stability and pharmacokinetics, easy to produce, and can be safely used for enhancing or assisting in vitro fertilization or embryo transfer process.

### DISCLOSURE OF THE INVENTION

The present inventors have unexpectedly found that a compound having a nonpeptidic gonadotropin releasing hormone antagonistic action is, based on its specific chemical structure, useful as a premature ovulation inhibitor for in vitro fertilization or embryo transfer process, or a premature ovulation inhibitor under controlled ovarian stimulation, and can be administered orally. Further studies based on these findings resulted in the completion of the present invention.

Accordingly, the present invention relates to

[1] a premature ovulation inhibitor for use in in vitro fertilization or embryo transfer process, comprising a nonpeptidic compound having a gonadotropin releasing hormone antagonistic action,
[2] the inhibitor of the above-mentioned [1], wherein the compound is represented by the formula

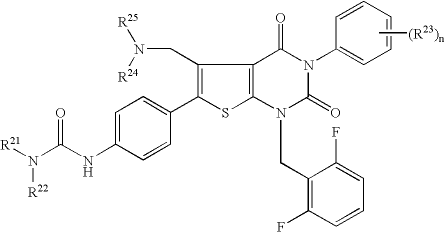

wherein R²¹ and R²² are each (1) a hydrogen atom, (2) hydroxy, (3) C₁₋₄ alkoxy, (4) C₁₋₄ alkoxy-carbonyl or (5) C₁₋₄ alkyl optionally having substituent(s),
R²³ is (1) a hydrogen atom, (2) a halogen atom, (3) hydroxy or (4) C₁₋₄ alkoxy optionally having substituent(s), or the adjacent two R²³ are optionally bonded to form C₁₋₄ alkylenedioxy,
R²⁴ is (1) a hydrogen atom or (2) C₁₋₄ alkyl, and
R²⁶ is (1) C₁₋₄ alkyl optionally having substituent(s) or (2) a group represented by the formula

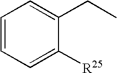

wherein R²⁵ is a hydrogen atom, or optionally bonded to R²⁴ to form heterocycle, and n is an integer of 0 to 5, or a salt thereof or a prodrug thereof,
[3] the inhibitor of the above-mentioned [1], wherein the compound is represented by the formula

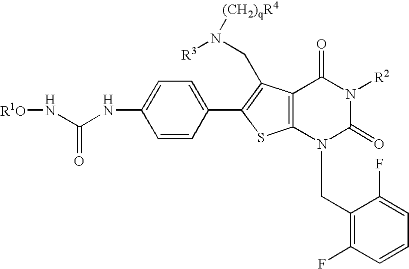

wherein
R¹ is C₁₋₄ alkyl,

### R² is

(1) C₁₋₆ alkyl optionally having substituent(s) selected from (1′) a hydroxyl group, (2′) C₁₋₄ alkoxy, (3′) C₁₋₄ alkoxy-carbonyl, (4′) di-C₁₋₄ alkyl-carbamoyl, (5′) a 5- to 7-membered nitrogen-containing heterocyclic group, (6′) C₁₋₄ alkyl-carbonyl and (7′) halogen,
(2) C₃₋₈ cycloalkyl optionally having (1′) a hydroxyl group or (2′) mono-C₁₋₄ alkyl-carbonylamino,
(3) a 5- to 7-membered nitrogen-containing heterocyclic group optionally having substituent(s) selected from (1′) halogen, (2′) a hydroxyl group, (3′) C₁₋₄ alkyl and (4′) C₁₋₄ alkoxy,
(4) phenyl optionally having substituent(s) selected from (1′) halogen, (2′) C₁₋₄ alkoxy-C₁₋₄ alkyl, (3′) mono-C₁₋₄ alkyl-carbamoyl-C₁₋₄ alkyl, (4′) C₁₋₄ alkoxy and (5′) mono-C₁₋₄ alkylcarbamoyl-C₁₋₄ alkoxy or
(5) C₁₋₄ alkoxy,
R³ is C₁₋₄ alkyl,
R⁴ is (1) a hydrogen atom, (2) C₁₋₄ alkoxy, (3) C₆₋₁₀ aryl, (4) N—C₁₋₄ alkyl-N—C₁₋₄ alkylsulfonylamino, (5) a hydroxyl group or
(6) a 5- to 7-membered nitrogen-containing heterocyclic group optionally having substituent(s) selected from (1′) oxo, (2′) C₁₋₄ alkyl, (3′) hydroxy-C₁₋₄ alkyl, (4′) C₁₋₄ alkoxy-carbonyl, (5′) mono-C₁₋₄ alkyl-carbamoyl and (6′) C₁₋₄ alkylsulfonyl,
q is an integer of 1 to 4,
(provided that when R² is phenyl optionally having substituent(s), R⁴ should be a 5- to 7-membered nitrogen-containing heterocyclic group optionally having substituent(s) selected from (1) oxo, (2) hydroxy-C₁₋₄ alkyl, (3) C₁₋₄ alkoxy-carbonyl, (4) mono-C₁₋₄ alkyl-carbamoyl and (5) C₁₋₄ alkylsulfonyl) or a salt thereof or a prodrug thereof,
[4] the inhibitor of any one of the above-mentioned [1] to [3], which is an oral preparation,
[5] a method of inhibiting premature ovulation in in vitro fertilization or embryo transfer process, which comprises administering an effective amount of the compound of the above-mentioned [1] to a mammal, and
[6] use of the compound of the above-mentioned [1] for the production of a premature ovulation inhibitor for in vitro fertilization or embryo transfer process.

The “nonpeptidic compound having a gonadotropin releasing hormone (GnRH) antagonistic action” (GnRH antagonist) of the present invention may be any as long as it is a nonpeptidic compound having a gonadotropin releasing hormone antagonistic action.

Examples of the nonpeptidic compound having a GnRH antagonistic action include a compound having a molecular weight of 1,000 or below, preferably 900 or below, more preferably 800 or below.

The compound preferably shows the property of superior oral absorbability. For example, a compound showing absorptivity of 10% of above by oral administration to a mammal at 10 mg/kg is preferable, and a compound showing absorptivity of 20% of above by oral administration to a mammal at 10 mg/kg is more preferable.

In addition, the compound preferably shows the property of permeability into the brain.

As the nonpeptidic compound having a GnRH antagonistic action, a fused heterocyclic compound meeting the above-mentioned conditions is particularly preferable.

As such fused heterocyclic compound, for example, a compound represented by the formula

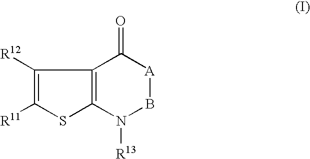

wherein R¹¹ is (1) a hydrogen atom, (2) a group via a carbon atom, (3) a group via a nitrogen atom, (4) a group via an oxygen atom or (5) a group via a sulfur atom, R¹² is (1) a hydrogen atom, (2) a group via a carbon atom, (3) a group via a nitrogen atom, (4) a group via an oxygen atom or (5) a group via a sulfur atom, R¹³ is (1) a hydrogen atom, (2) alkyl or (3) —(CH₂)ₚQ (p is an integer of 0 to 3, Q is a homocyclic group optionally having substituent(s) or a heterocyclic group optionally having substituent(s)),

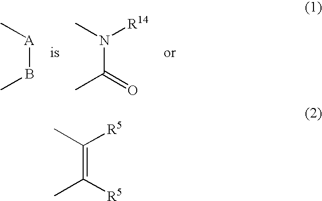

wherein R¹⁴ is (1) a hydrogen atom, (2) alkyl optionally having alkoxy, (3) aryl optionally having substituent(s), (4) aralkyl optionally having substituent(s) or (5) cycloalkyl optionally having substituent(s), R⁵ is (1) a hydrogen atom, (2) formyl, (3) cyano, (4) C₁₋₆ alkyl optionally having (i) a group via a sulfur atom or (ii) a group via an oxygen atom, (5) a heterocyclic group optionally having substituent(s), (6) a group via a nitrogen atom, (7) a group via an oxygen atom, (8) a group via a sulfur atom, (9) carboxyl optionally esterified, thioesterified or amidated or (10) —C(O)R⁷ (R⁷ is a hydrocarbon group optionally having substituent(s), and R⁶ is (1) a hydrogen atom or (2) a group via a carbon atom (hereinafter sometimes to be abbreviated as compound (I)) or a salt thereof or a prodrug thereof and the like.

The definition of each substituent in the aforementioned compound (I) is shown below.

Examples of the “group via a carbon atom” for R¹¹, R¹² or R⁶ include (1) alkyl optionally having substituent(s), (2) cycloalkyl optionally having substituent(s), (3) alkenyl optionally having substituent(s), (4) aryl optionally having substituent(s), (5) aralkyl optionally having substituent(s), (6) a heterocyclic group having a bond at a carbon atom (the heterocyclic group optionally has substituent(s)), (7) formyl, (8) optionally esterified or amidated carboxyl, (9) cyano, (10) amidino and the like.

Examples of the alkyl of the “alkyl optionally having substituent(s)” defined as the “group via a carbon atom” for R¹¹, R¹² or R⁶ include linear or branched C₁₋₆ alkyl such as methyl, ethyl, propyl, isopropyl, butyl, isobutyl, sec-butyl, tert-butyl, pentyl, isopentyl, neopentyl, hexyl, isohexyl, 3-methylpentyl, neohexyl, 2,3-dimethylbutyl and the like, and the like.

Examples of the substituent of the “alkyl optionally having substituent(s)” include (1) C₆₋₁₄ aryl (e.g., phenyl, naphthyl etc.) optionally having 1 to 4 substituents selected from (i) hydroxy, (ii) amino, (iii) mono- or di-C₁₋₆ alkylamino (e.g., methylamino, ethylamino, propylamino, dimethylamino, diethylamino etc.), (iv) C₁₋₆ alkoxy (e.g., methoxy, ethoxy, propoxy, butoxy, pentoxy, hexyloxy etc.) and (v) a halogen atom (e.g., fluorine, chlorine, bromine, iodine), (2) hydroxy, (3) carboxy, (4) nitro, (5) C₁₋₆ alkoxy (e.g., methoxy, ethoxy, propoxy, isopropoxy, butoxy, pentoxy, hexyloxy etc.), (6) C₁₋₆ alkyl-carbonyloxy (e.g., acetoxy, propionyloxy, butyryloxy, isobutyryloxy, valeryloxy, isovaleryloxy, pivaloyloxy, pentylcarbonyloxy, hexylcarbonyloxy etc.), (7) C₁₋₆ alkylthio (e.g., methylthio, ethylthio, propylthio, isopropylthio, butylthio, isobutylthio, sec-butylthio, tert-butylthio, pentylthio, hexylthio etc.), (8) C₁₋₆ alkylsulfinyl (e.g., methylsulfinyl, ethylsulfinyl, propylsulfinyl, isopropylsulfinyl, butylsulfinyl, isobutylsulfinyl, sec-butylsulfinyl, tert-butylsulfinyl, pentylsulfinyl, hexylsulfinyl etc.), (9) C₁₋₆ alkylsulfonyl (e.g., methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl, hexylsulfonyl etc.), (10) a halogen atom (e.g., fluorine, chlorine, bromine, iodine), (11) a group via a nitrogen atom, (12) a heterocyclic group and the like.

Examples of the “group via a nitrogen atom” as the substituent of the “alkyl optionally having substituent(s)” include (1) —NR⁸OR⁹ wherein R⁸ is a hydrogen atom, C₁₋₆ alkyl optionally having substituent(s), C₃₋₆ cycloalkyl optionally having substituent(s), C₆₋₁₄ aryl optionally having substituent(s), C₇₋₂₀ aralkyl optionally having substituent(s), acyl, carbamoyl optionally having substituent(s) or a heterocyclic group, R⁹ is a hydrogen atom or C₁₋₆ alkyl optionally having substituent(s), (2) a heterocyclic group having a bond at a nitrogen atom (e.g., 1H-1-pyrrolyl, 1-imidazolyl, pyrazolyl, indolyl, 1H-1-indazolyl, 7-purinyl, 1-pyrrolidinyl, 1-pyrrolinyl, 1-imidazolidinyl, pyrazolidinyl, piperazinyl, pyrazolinyl, 1-piperidinyl, 4-morpholinyl, 4-thiomorpholinyl, 2-isoindolyl, 2-(1,2,3,4-tetrahydro)isoquinolyl etc.) and the like.

Examples of the C₁₋₆ alkyl of the “C₁₋₆ alkyl optionally having substituent(s)” for R⁸ or R⁹ include methyl, ethyl, propyl, isopropyl, butyl, isobutyl, sec-butyl, tert-butyl, pentyl, isopentyl, neopentyl, hexyl, isohexyl, 3-methylpentyl, neohexyl, 2,3-dimethylbutyl and the like.

Examples of the substituent of the “C₁₋₆ alkyl optionally having substituent(s)” for R⁸ or R⁹ include (1) C₁₋₆ alkyl (e.g., methyl, ethyl, propyl, isopropyl, butyl, isobutyl, sec-butyl, tert-butyl, pentyl, isopentyl, neopentyl, hexyl, isohexyl, 3-methylpentyl, neohexyl, 2,3-dimethylbutyl etc.), (2) C₂₋₆ alkenyl (e.g., vinyl, 1-methylvinyl, 1-propenyl, allyl etc.), (3) C₂₋₆ alkynyl (e.g., ethynyl, 1-propynyl, propargyl etc.), (4) C₃₋₆ cycloalkyl (e.g., cyclopropyl, cyclobutyl, cyclopentyl, cyclohexyl etc.), (5) C₅₋₇ cycloalkenyl (e.g., cyclopentenyl, cyclohexenyl etc.), (6) C₇₋₁₁ aralkyl (e.g., benzyl, α-methylbenzyl, phenethyl etc.), (7) C₆₋₁₄ aryl (e.g., phenyl, naphthyl etc.), (8) C₁₋₆ alkoxy (e.g., methoxy, ethoxy, propoxy, isopropoxy, butoxy, isobutoxy, sec-butoxy, tert-butoxy etc.), (9) C₆₋₁₄ aryloxy (e.g., phenoxy, 1-naphthoxy, 2-naphthoxy etc.), (10) C₁₋₆ alkanoyl (e.g., formyl, acetyl, propionyl, butyryl, isobutyryl etc.), (11) C₆₋₁₄ aryl-carbonyl (e.g., benzoyl, 1-naphthylcarbonyl, 2-naphthylcarbonyl etc.), (12) C₁₋₆ alkanoyloxy (e.g., formyloxy, acetoxy, propionyloxy, butyryloxy, isobutyryloxy etc.), (13) C₆₋₁₄ aryl-carbonyloxy (e.g., benzoyloxy, 1-naphthylcarbonyloxy, 2-naphthylcarbonyloxy etc.), (14) carboxy, (15) C₁₋₆ alkoxy-carbonyl (e.g., methoxycarbonyl, ethoxycarbonyl, propoxycarbonyl, isopropoxycarbonyl, butoxycarbonyl, isobutoxycarbonyl, tert-butoxycarbonyl etc.), (16) carbamoyl, (17) N-mono-C₁₋₄ alkylcarbamoyl (e.g., N-methylcarbamoyl, N-ethylcarbamoyl, N-propylcarbamoyl, N-isopropylcarbamoyl, N-butylcarbamoyl etc.), (18) N,N-di-C₁₋₄ alkylcarbamoyl (e.g., N,N-dimethylcarbamoyl, N,N-diethylcarbamoyl, N,N-dipropylcarbamoyl, N,N-dibutylcarbamoyl etc.), (19) cyclic aminocarbonyl (e.g., 1-aziridinylcarbonyl, 1-azetidinylcarbonyl, 1-pyrrolidinylcarbonyl, 1-piperidinylcarbonyl, N-methylpiperazinylcarbonyl, morpholinocarbonyl etc.), (20) a halogen atom (e.g., fluorine, chlorine, bromine, iodine), (21) C₁₋₄ alkyl substituted by 1 to 3 halogen atoms (e.g., chloromethyl, dichloromethyl, trifluoromethyl, trifluoroethyl etc.), (22) oxo, (23) amidino, (24) imino, (25) amino, (26) mono- or di-C₁₋₄ alkylamino (e.g., methylamino, ethylamino, propylamino, isopropylamino, butylamino, isobutylamino, sec-butylamino, tert-butylamino, pentylamino, hexylamino, dimethylamino, diethylamino, dipropylamino etc.), (27) 3 to 6-membered cyclic amino (e.g., aziridinyl, azetidinyl, pyrrolidinyl, pyrrolinyl, pyrrolyl, imidazolyl, pyrazolyl, imidazolidinyl, piperidino, morpholino, dihydropyridyl, pyridyl, N-methylpiperazinyl, N-ethylpiperazinyl etc.) optionally containing, besides carbon atom(s) and one nitrogen atom, 1 to 3 hetero atoms selected from an oxygen atom, a sulfur atom, a nitrogen atom and the like, (28) C₁₋₆ alkanoylamino (e.g., formylamino, acetylamino, trifluoroacetylamino, propionylamino, butyrylamino, isobutyrylamino etc.), (29) benzamido, (30) carbamoylamino, (31) (N—C₁₋₄ alkylcarbamoyl)amino (e.g., (N-methylcarbamoyl)amino, (N-ethylcarbamoyl)amino, (N-propylcarbamoyl)amino, (N-isopropylcarbamoyl)amino, (N-butylcarbamoyl)amino etc.), (32) (N,N-di-C₁₋₄ alkylcarbamoyl)amino (e.g., (N,N-dimethylcarbamoyl)amino, (N,N-diethylcarbamoyl)amino, (N,N-dipropylcarbamoyl)amino, (N,N-dibutylcarbamoyl)amino etc.), (33) C₁₋₆ alkylenedioxy (e.g., —OCH₂O—, —O(CH₂)₂O—, —O(CH₂)₃O—, —O(CH₂)₄O—, —O(CH₂)₅O—, —O(CH₂)₆O— etc.), (34) dihydroboryl, (35) hydroxy, (36) epoxy, (37) nitro, (38) cyano, (39) mercapto, (40) sulfo, (41) sulfino, (42) phosphono, (43) sulfamoyl, (44) N—C₁₋₆ alkylsulfamoyl (e.g., N-methylsulfamoyl, N-ethylsulfamoyl, N-propylsulfamoyl, N-isopropylsulfamoyl, N-butylsulfamoyl etc.), (45) N,N-diC₁₋₆ alkylsulfamoyl (e.g., N,N-dimethylsulfamoyl, N,N-diethylsulfamoyl, N,N-dipropylsulfamoyl, N,N-dibutylsulfamoyl etc.), (46) C₁₋₆ alkylthio (e.g., methylthio, ethylthio, propylthio, isopropylthio, butylthio, sec-butylthio, tert-butylthio etc.), (47) phenylthio, (48) C₁₋₆ alkylsulfinyl (e.g., methylsulfinyl, ethylsulfinyl, propylsulfinyl, butylsulfinyl etc.), (49) phenylsulfinyl, (50) C₁₋₆ alkylsulfonyl (e.g., methylsulfonyl, ethylsulfonyl, propylsulfonyl, butylsulfonyl etc.), (51) phenylsulfonyl and the like. In addition, 1 to 6, preferably 1 to 3, substituents selected therefrom may be present at substitutable position(s).

Examples of the C₃₋₆ cycloalkyl of the “C₃₋₆ cycloalkyl optionally having substituent(s)” for R⁸ include cyclopropyl, cyclobutyl, cyclopentyl, cyclohexyl and the like.

Examples of the substituent of the “C₃₋₆ cycloalkyl optionally having substituent(s)” for R⁸ include those similar to the substituents of the aforementioned “C₁₋₆ alkyl optionally having substituent(s)” for R⁸ or R⁹, where 1 to 6, preferably 1 to 3, substituents may be present at substitutable position(s).

Examples of the C₆₋₁₄ aryl of the “optionally having substituent(s) C₆₋₁₄ aryl” for R⁸ include phenyl, naphthyl, anthracenyl and the like.

Examples of the substituent of the “C₆₋₁₄ aryl optionally having substituent(s)” for R⁸ include the substituents of the aforementioned “C₁₋₆ alkyl optionally having substituent(s)” for R⁸ or R⁹ less oxo and epoxy, where 1 to 6, preferably 1 to 3, substituents may be present at substitutable position(s).

Examples of C₇₋₂₀ aralkyl of the “C₇₋₂₀ aralkyl optionally having substituent(s)” for R⁸ include benzyl, phenethyl, phenylpropyl, benzhydryl, trityl and the like.

Examples of the substituent of the “C₇₋₂₀ aralkyl optionally having substituent(s)” for R⁸ include those similar to the substituents of the aforementioned “C₁₋₆ alkyl optionally having substituent(s)” for R⁸ or R⁹, where 1 to 6, preferably 1 to 3, substituents may be present at substitutable position(s).

Examples of the “acyl” for R⁸ include groups formed by the aforementioned “C₁₋₆ alkyl optionally having substituent(s)”, “C₃₋₆ cycloalkyl optionally having substituent(s)”, “C₆₋₁₄ aryl optionally having substituent(s)” or “C₇₋₂₀ aralkyl optionally having substituent(s)” for R⁸ and carbonyl, sulfinyl or sulfonyl bonded to each other and the like.

Examples of the substituent of the “carbamoyl optionally having substituent(s)” for R⁸ include (1) C₁₋₆ alkyl optionally having substituent(s), (2) C₃₋₆ cycloalkyl optionally having substituent(s), (3) C₆₋₁₄ aryl optionally having substituent(s), (4) C₇₋₂₀ aralkyl optionally having substituent(s), (5) hydroxy, (6) C₁₋₆ alkoxy optionally having substituent(s) and (7) C₁₋₆ alkoxy-carbonyl optionally having substituent(s) and the like, where 1 or 2 substituents therefrom may be present.

Examples of the “C₁₋₆ alkyl optionally having substituent(s)” as the substituent of the “carbamoyl optionally having substituent(s)” for R⁸ include those similar to the aforementioned “C₁₋₆ alkyl optionally having substituent(s)” for R⁸ or R⁹.

Examples of the “C₃₋₆ cycloalkyl optionally having substituent(s)”, “C₆₋₁₄ aryl optionally having substituent(s)” and “C₇₋₂₀ aralkyl optionally having substituent(s)” as the substituent of the “carbamoyl optionally having substituent(s)” for R⁸ include those similar to the aforementioned “C₃₋₆ cycloalkyl optionally having substituent(s)”, “C₆₋₁₄ aryl optionally having substituent(s)” and “C₇₋₂₀ aralkyl optionally having substituent(s)” for R⁸.

Examples of the C₁₋₆ alkoxy of the “C₁₋₆ alkoxy optionally having substituent(s)” as the substituent of the “carbamoyl optionally having substituent(s)” for R⁸ include methoxy, ethoxy, propoxy, isopropoxy, butoxy, isobutoxy, sec-butoxy, tert-butoxy, pentoxy, hexyloxy and the like. Examples of the substituent of the “C₁₋₆ alkoxy optionally having substituent(s)” include those similar to the substituents of the aforementioned “C₁₋₆ alkyl optionally having substituent(s)” for R⁸, where 1 to 6, preferably 1 to 3, substituents may be present at substitutable position(s).

Examples of the “C₁₋₆ alkoxy optionally having substituent(s)-carbonyl” as the substituent of the “carbamoyl optionally having substituent(s)” for R⁸ include a group formed by the “C₁₋₆ alkoxy optionally having substituent(s)” as the substituent of the aforementioned “carbamoyl optionally having substituent(s)” for R⁸ and carbonyl bonded to each other.

Examples of the “heterocyclic group” for R⁸ include (1) a 5-membered cyclic group containing, besides carbon atom(s), 1 to 4 hetero atoms selected from an oxygen atom, a sulfur atom, a nitrogen atom and the like (e.g., 2-thienyl, 3-thienyl, 2-furyl, 3-furyl, 2-pyrrolyl, 3-pyrrolyl, 2-oxazolyl, 4-oxazolyl, 5-oxazolyl, 2-thiazolyl, 4-thiazolyl, 5-thiazolyl, 3-pyrazolyl, 4-pyrazolyl, 5-pyrazolyl, 2-imidazolyl, 4-imidazolyl, 5-imidazolyl, 3-isoxazolyl, 4-isoxazolyl, 5-isoxazolyl, 3-isothiazolyl, 4-isothiazolyl, 5-isothiazolyl, 3-(1,2,4-oxadiazolyl), 5-(1,2,4-oxadiazolyl), 1,3,4-oxadiazolyl, 3-(1,2,4-thiadiazolyl), 5-(1,2,4-thiadiazolyl), 1,3,4-thiadiazolyl, 4-(1,2,3-thiadiazolyl), 5-(1,2,3-thiadiazolyl), 1,2,5-thiadiazolyl, 1,2,3-triazolyl, 1,2,4-triazolyl, 1H-tetrazolyl, 2H-tetrazolyl, oxoimidazinyl, dioxotriazinyl, pyrrolidinyl etc.), (2) a 6-membered cyclic group containing, besides carbon atom(s), 1 to 4 hetero atoms selected from an oxygen atom, a sulfur atom, a nitrogen atom and the like (e.g., 2-pyridyl, 3-pyridyl, 4-pyridyl, N-oxido-2-pyridyl, N-oxido-3-pyridyl, N-oxido-4-pyridyl, 2-pyrimidinyl, 4-pyrimidinyl, 5-pyrimidinyl, N-oxido-2-pyrimidinyl, N-oxido-4-pyrimidinyl, N-oxido-5-pyrimidinyl, 2-thiomorpholinyl, 3-thiomorpholinyl, 2-morpholinyl, 3-morpholinyl, piperidinyl, pyranyl, thiopyranyl, 1,4-oxazinyl, 1,4-thiazinyl, 1,3-thiazinyl, 2-piperazinyl, 3-piperazinyl, triazinyl, oxotriazinyl, 3-pyridazinyl, 4-pyridazinyl, pyrazinyl, N-oxido-3-pyridazinyl, N-oxido-4-pyridazinyl etc.), and (3) a bicyclic or tricyclic fused ring group containing, besides carbon atom(s), 1 to 4 hetero atoms selected from an oxygen atom, a sulfur atom, a nitrogen atom and the like (e.g., benzofuryl, benzothiazolyl, benzoxazolyl, tetrazolo[1,5-b]pyridazinyl, triazolo[4,5-b]pyridazinyl, benzimidazolyl, quinolyl, isoquinolyl, cinnolinyl, phthalazinyl, quinazolinyl, quinoxalinyl, indolizinyl, quinolizinyl, 1,8-naphthyridinyl, purinyl, pteridinyl, dibenzofuranyl, carbazolyl, acrydinyl, phenanthridinyl, chromanyl, benzoxazinyl, phenazinyl, phenothiazinyl, phenoxazinyl etc.).

Examples of the heterocyclic group as a substituent of the “alkyl optionally having substituent(s)” defined as the “group via a carbon atom” for R¹¹, R¹² or R⁶ include those similar to the aforementioned “heterocyclic group” for R⁸.

Examples of the cycloalkyl of the “cycloalkyl optionally having substituent(s)” defined as the “group via a carbon atom” for R¹¹, R¹² or R⁶ include C₃₋₆ cycloalkyl such as cyclopropyl, cyclobutyl, cyclopentyl, cyclohexyl and the like, and the like.

Examples of the substituent of the “cycloalkyl optionally having substituent(s)” include those similar to the substituent of the “alkyl optionally having substituent(s)” defined as the “group via a carbon atom” for R¹¹, R¹² or R⁶, where 1 to 6, preferably 1 to 3, substituents may be present at substitutable position(s).

Examples of the alkenyl of the “alkenyl optionally having substituent(s)” defined as the “group via a carbon atom” for R¹¹, R¹² or R⁶ include C₂₋₆ alkenyl such as vinyl, butadienyl, hexatrienyl and the like, and the like.

Examples of the substituent of the alkenyl of the “alkenyl optionally having substituent(s)” for R¹¹, R¹² or R⁶ include those similar to the substituent of the “alkyl optionally having substituent(s)” defined as the “group via a carbon atom”, where 1 to 6, preferably 1 to 3, substituents may be present at substitutable position(s).

Examples of the aryl of the “aryl optionally having substituent(s)” defined as the “group via a carbon atom” for R¹¹, R¹² or R⁶ include C₆₋₁₄ aryl such as phenyl, naphthyl, anthracenyl and the like, and the like.

Examples of the substituent of aryl of the “aryl optionally having substituent(s)” include those similar to the substituent of the “alkyl optionally having substituent(s)” defined as the “group via a carbon atom” for R¹¹, R¹² or R⁶, such as C₁₋₆ alkoxycarbonyl (e.g., methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, hexyloxycarbonyl etc.), carbamoyl, N-mono-C₁₋₆ alkylcarbamoyl (e.g., N-methylcarbamoyl, N-ethylcarbamoyl, N-propylcarbamoyl, N-isopropylcarbamoyl etc.), N,N-di-C₁₋₆ alkylcarbamoyl (e.g., N,N-dimethylcarbamoyl, N,N-diethylcarbamoyl, N,N-dipropylcarbamoyl etc.) and the like, where 1 to 6, preferably 1 to 3, substituents may be present at substitutable position(s).

Examples of aralkyl of the “aralkyl optionally having substituent(s)” defined as the “group via a carbon atom” for R¹¹, R¹² or R⁶ include C₇₋₂₀ aralkyl such as benzyl, benzhydryl, trityl and the like, and the like.

Examples of the substituent of aralkyl of the “aralkyl optionally having substituent(s)” include those similar to the substituents of the “alkyl optionally having substituent(s)” defined as the “group via a carbon atom” for R¹¹, R¹² or R⁶, where 1 to 6, preferably 1 to 3, substituents may be present at substitutable position(s).

Examples of the “heterocyclic group having a bond at a carbon atom” defined as the “group via a carbon atom” for R¹¹, R¹² or R⁶ include those similar to the heterocyclic group for R⁸.

The “heterocyclic group having a bond at a carbon atom” may have a substituent. Examples of the substituent include those similar to the substituents of the “alkyl optionally having substituent(s)” defined as the “group via a carbon atom” for R¹¹, R¹² or R⁶, where 1 to 6, preferably 1 to 3, substituents may be present at substitutable position(s).

Examples of the “optionally esterified carboxyl” defined as the “group via a carbon atom” for R¹¹, R¹² or R⁶ include a group represented by —CO₂R¹⁰ (R¹⁰ is hydrogen, alkyl optionally having substituent(s), cycloalkyl optionally having substituent(s), aryl optionally having substituent(s), aralkyl optionally having substituent(s) or a heterocyclic group having a bond at a carbon atom (the heterocyclic group optionally has substituent(s)).

Examples of the “alkyl optionally having substituent(s)”, “cycloalkyl optionally having substituent(s)”, “aryl optionally having substituent(s)”, “aralkyl optionally having substituent(s)”, “heterocyclic group having a bond at a carbon atom (the heterocyclic group optionally has substituent(s))” for R¹⁰ include those similar to the “alkyl optionally having substituent(s)”, “cycloalkyl optionally having substituent(s)”, “aryl optionally having substituent(s)”, “aralkyl optionally having substituent(s)” and “heterocyclic group having a bond at a carbon atom (the heterocyclic group optionally has substituent(s))” as the aforementioned “group via a carbon atom” for R¹¹, R¹² or R⁶.

Examples of the “optionally amidated carboxyl” defined as the “group via a carbon atom” for R¹¹, R¹² or R⁶ include a group represented by —CONR⁸R⁹ (R⁸ and R⁹ are as defined above).

Examples of the “group via a nitrogen atom” for R¹¹, R¹² or R⁵ include those similar to the “group via a nitrogen atom”, which is a substituent of the “alkyl optionally having substituent(s)” defined as the “group via a carbon atom” for R¹¹, R¹² or R⁶.

Examples of the “group via an oxygen atom” for R¹¹, R¹² or R⁵ include a group represented by —OR¹⁵ (R¹⁵ is C₁₋₆ alkyl optionally having substituent(s), C₃₋₆ cycloalkyl optionally having substituent(s), C₆₋₁₄ aryl optionally having substituent(s), C₇₋₂₀ aralkyl optionally having substituent(s) or a heterocyclic group optionally having substituent(s)).

Examples of the “C₁₋₆ alkyl optionally having substituent(s)” for R¹⁵ include those similar to the aforementioned “C₁₋₆ alkyl optionally having substituent(s)” for R⁸ or R⁹.

Examples of the “C₃₋₆ cycloalkyl optionally having substituent(s)”, “C₆₋₁₄ aryl optionally having substituent(s)”, “C₇₋₂₀ aralkyl optionally having substituent(s)” and “heterocyclic group optionally having substituent(s)” for R¹⁵ include those similar to the aforementioned “C₃₋₆ cycloalkyl optionally having substituent(s)”, “C₆₋₁₄ aryl optionally having substituent(s)”, “C₇₋₂₀ aralkyl optionally having substituent(s)” and “heterocyclic group optionally having substituent(s)” for R⁸.

Examples of the “group via a sulfur atom” for R¹¹, R¹² or R⁵ include a group represented by —SR¹⁵ (R¹⁵ is as defined above).

Examples of the alkyl for R¹³ include C₁₋₆ alkyl such as methyl, ethyl, propyl, isopropyl, butyl, isobutyl, sec-butyl, tert-butyl, pentyl, hexyl and the like, and the like.

Examples of the “homocyclic group optionally having substituent(s)” for Q include (1) aryl optionally having substituent(s) and (2) cycloalkyl optionally having substituent(s).

Examples of the aryl of the “aryl optionally having substituent(s)” as the definition of the “homocyclic group optionally having substituent(s)” for Q include C₆₋₁₄ aryl such as phenyl, 1-naphthyl, 2-naphthyl, anthryl, phenanthryl, acenaphthyl and the like, and the like.

Examples of the substituent of aryl of the “aryl optionally having substituent(s)” as the definition of the “homocyclic group optionally having substituent(s)” for Q include (i) C₁₋₆ alkyl (e.g., methyl, ethyl, propyl, isopropyl, butyl, isobutyl, sec-butyl, tert-butyl, pentyl, hexyl etc.), (ii) C₂₋₆ alkenyl (e.g., vinyl, allyl, 1-butenyl, 2-butenyl etc.), (iii) C₂₋₆ alkynyl (e.g., ethynyl, propargyl, 2-butynyl, 5-hexynyl etc.), (iv) C₃₋₆ cycloalkyl (e.g., cyclopropyl, cyclobutyl, cyclopentyl, cyclohexyl etc.), (v) C₆₋₁₄ aryl (e.g., phenyl, 1-naphthyl, 2-naphthyl etc.), (vi) C₇₋₁₄ aralkyl (e.g., benzyl, phenethyl etc.), (vii) nitro, (viii) hydroxy, (ix) mercapto, (x) cyano, (xi) carbamoyl, (xii) carboxyl, (xiii) C₁₋₆ alkoxy-carbonyl (e.g., methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, hexyloxycarbonyl etc.), (xiv) sulfo, (xv) a halogen atom (e.g., fluorine, chlorine, bromine, iodine), (xvi) C₁₋₆ alkoxy (e.g., methoxy, ethoxy, propoxy, isopropoxy, butoxy, isobutoxy, sec-butoxy, tert-butoxy, pentyloxy, hexyloxy etc.) optionally having C₁₋₆ alkoxy (e.g., methoxy, ethoxy, propoxy, isopropoxy, butoxy, isobutoxy, sec-butoxy, tert-butoxy, pentyloxy, hexyloxy etc.), (xvii) C₆₋₁₀ aryloxy (e.g., phenoxy, 1-naphthyloxy, 2-naphthyloxy etc.), (xviii) C₁₋₆ alkylthio (e.g., methylthio, ethylthio, propylthio, isopropylthio, butylthio, isobutylthio, sec-butylthio, tert-butylthio, pentylthio, hexylthio etc.), (xix) C₆₋₁₀ arylthio (e.g., phenylthio, 1-naphthylthio, 2-naphthylthio etc.), (xx) C₁₋₆ alkylsulfinyl (e.g., methylsulfinyl, ethylsulfinyl, propylsulfinyl, isopropylsulfinyl, butylsulfinyl, isobutylsulfinyl, sec-butylsulfinyl, tert-butylsulfinyl, pentylsulfinyl, hexylsulfinyl etc.), (xxi) C₆₋₁₀ arylsulfinyl (e.g., phenylsulfinyl, 1-naphthylsulfinyl, 2-naphthylsulfinyl etc.), (xxii) C₁₋₆ alkylsulfonyl (e.g., methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl, hexylsulfonyl etc.), (xxiii) C₆₋₁₀ arylsulfonyl (e.g., phenylsulfonyl, 1-naphthylsulfonyl, 2-naphthylsulfonyl etc.), (xxiv) amino, (xxv) C₁₋₆ acylamino (e.g., formylamino, acetylamino, propionylamino, butyrylamino, isobutyrylamino, valerylamino etc.), (xxvi) mono-C₁₋₆ alkylamino (e.g., methylamino, ethylamino, propylamino, isopropylamino, butylamino etc.), (xxvii) di-C₁₋₆ alkylamino (e.g., dimethylamino, diethylamino, dipropylamino, diisopropylamino, dibutylamino etc.), (xxviii) C₃₋₆ cycloalkylamino (e.g., cyclopropylamino, cyclobutylamino, cyclopentylamino, cyclohexylamino etc.), (xxix) C₆₋₁₀ arylamino (e.g., anilino, 1-naphthylamino, 2-naphthylamino etc.), (xxx) C₁₋₆ acyl (e.g., formyl, acetyl, propionyl, butyryl, isobutyryl, valeryl etc.), (xxxi) C₆₋₁₀ arylcarbonyl (e.g., benzoyl, 1-naphthylcarbonyl, 2-naphthylcarbonyl etc.), (xxxii) C₁₋₄ alkylenedioxy (e.g., —OCH₂O—, —O(CH₂)₂O—, —O(CH₂)₃O—, —O(CH₂)₄O—), (xxxiii) a 5 or 6-membered heterocyclic group containing, besides carbon atom(s), 1 to 4 hetero atoms selected from an oxygen atom, a sulfur atom, a nitrogen atom and the like (e.g., 2-thienyl, 3-thienyl, 2-furyl, 3-furyl, 2-pyrrolyl, 3-pyrrolyl, 2-oxazolyl, 4-oxazolyl, 5-oxazolyl, 2-thiazolyl, 4-thiazolyl, 5-thiazolyl, 3-pyrazolyl, 4-pyrazolyl, 5-pyrazolyl, 2-imidazolyl, 4-imidazolyl, 5-imidazolyl, 3-isoxazolyl, 4-isoxazolyl, 5-isoxazolyl, 3-isothiazolyl, 4-isothiazolyl, 5-isothiazolyl, 3-(1,2,4-oxadiazolyl), 5-(1,2,4-oxadiazolyl), 1,3,4-oxadiazolyl, 3-(1,2,4-thiadiazolyl), 5-(1,2,4-thiadiazolyl), 1,3,4-thiadiazolyl, 4-(1,2,3-thiadiazolyl), 5-(1,2,3-thiadiazolyl), 1,2,5-thiadiazolyl, 1,2,3-triazolyl, 1,2,4-triazolyl, 1H-tetrazolyl, 2H-tetrazolyl, oxoimidazinyl, dioxotriazinyl, pyrrolidinyl, 2-pyridyl, 3-pyridyl, 4-pyridyl, 2-pyrimidinyl, 4-pyrimidinyl, 5-pyrimidinyl, 2-thiomorpholinyl, 3-thiomorpholinyl, 2-morpholinyl, 3-morpholinyl, piperidinyl, pyranyl, thiopyranyl, 1,4-oxazinyl, 1,4-thiazinyl, 1,3-thiazinyl, 2-piperazinyl, 3-piperazinyl, triazinyl, oxotriazinyl, 3-pyridazinyl, 4-pyridazinyl, pyrazinyl etc.) and the like, where 1 to 6, preferably 1 to 3, substituents may be present at substitutable position(s).

Examples of the cycloalkyl of the “cycloalkyl optionally having substituent(s)” as the definition of the “homocyclic group optionally having substituent(s)” for Q include C₃₋₆ cycloalkyl such as cyclopropyl, cyclobutyl, cyclopentyl, cyclohexyl and the like, and the like.

Examples of the substituent of cycloalkyl of the “cycloalkyl optionally having substituent(s)” as the definition of the “homocyclic group optionally having substituent(s)” for Q include those similar to the substituent of the “aryl optionally having substituent(s)” as the definition of the aforementioned “homocyclic group optionally having substituent(s)” for Q, oxo, thioxo and the like, where 1 to 6, preferably 1 to 3, substituents may be present at substitutable position(s).

Examples of the heterocyclic group of the “heterocyclic group optionally having substituent(s)” for Q include those similar to the aforementioned “heterocyclic group” for R⁸.

Examples of the substituent of the heterocyclic group of the “heterocyclic group optionally having substituent(s)” for Q include those similar to the substituent of aryl of the “aryl optionally having substituent(s)” as the definition of the aforementioned “homocyclic group optionally having substituent(s)” for Q, where 1 to 6, preferably 1 to 3, substituents may be present at substitutable position(s).

Examples of the “alkyl” of the “alkyl optionally having alkoxy” for R¹⁴ include C₁₋₆ alkyl such as methyl, ethyl, propyl, isopropyl, butyl, isobutyl, sec-butyl, tert-butyl, pentyl, isopentyl, neopentyl, hexyl, isohexyl, 3-methylpentyl, neohexyl, 2,3-dimethylbutyl and the like, and the like.

Examples of the “alkoxy” of the “alkyl optionally having alkoxy” for R¹⁴ include C₁₋₆ alkoxy such as methoxy, ethoxy, propoxy, isopropoxy, butoxy, isobutoxy, sec-butoxy, tert-butoxy, pentyloxy, hexyloxy and the like, and the like.

Examples of the “aryl” of the “aryl optionally having substituent(s)” for R¹⁴ include C₆₋₁₄ aryl such as phenyl, 1-naphthyl, 2-naphthyl and the like, and the like.

Examples of the substituent of the “aryl optionally having substituent(s)” for R¹⁴ include those similar to the substituent of aryl of the “aryl optionally having substituent(s)” as the definition of the aforementioned “homocyclic group optionally having substituent(s)” for Q, where 1 to 6, preferably 1 to 3, substituents may be present at substitutable position(s).

Examples of the aralkyl of the “aralkyl optionally having substituent(s)” for R¹⁴ include C₇₋₂₀ aralkyl such as benzyl, benzhydryl, trityl and the like, and the like.

Examples of the substituent of the “aralkyl optionally having substituent(s)” for R¹⁴ include those similar to the substituent of aryl of the “aryl optionally having substituent(s)” as the definition of the aforementioned “homocyclic group optionally having substituent(s)” for Q, where 1 to 6, preferably 1 to 3, substituents may be present at substitutable position(s).

Examples of the cycloalkyl of the “cycloalkyl optionally having substituent(s)” for R¹⁴ include C₃₋₆ cycloalkyl such as cyclopropyl, cyclobutyl, cyclopentyl, cyclohexyl and the like, and the like.

Examples of the substituent of the “cycloalkyl optionally having substituent(s)” for R¹⁴ include those similar to the substituent of aryl of the “aryl optionally having substituent(s)” as the definition of the aforementioned “homocyclic group optionally having substituent(s)” for Q, where 1 to 6, preferably 1 to 3, substituents may be present at substitutable position(s).

Examples of the “C₁₋₆ alkyl” of “(i) a group via a sulfur atom or (ii) C₁₋₆ alkyl optionally having a group via an oxygen atom” for R⁵ include C₁₋₆ alkyl such as methyl, ethyl, propyl, isopropyl, butyl, isobutyl, sec-butyl, tert-butyl, pentyl, isopentyl, neopentyl, hexyl, isohexyl, 3-methylpentyl, neohexyl, 2,3-dimethylbutyl and the like, and the like.

Examples of the “group via a sulfur atom” of “(i) a group via a sulfur atom or (ii) C₁₋₆ alkyl optionally having a group via an oxygen atom” for R⁵ include a group similar to the aforementioned “group via a sulfur atom” for R¹¹ or R¹².

Examples of the “group via an oxygen atom” of “(i) a group via a sulfur atom or (ii) C₁₋₆ alkyl optionally having a group via an oxygen atom” for R⁵ include a group similar to the aforementioned “group via an oxygen atom” for R¹¹ or R¹².

Examples of the “heterocyclic group optionally having substituent(s)” for R⁵ include those similar to the aforementioned “heterocyclic group optionally having substituent(s)” for Q.

Examples of the “optionally esterified carboxyl” for R⁵ include a group represented by —CO₂R¹⁰ (R¹⁰ is as defined above).

Examples of the “optionally thioesterified carboxyl” for R⁵ include a group represented by —C(O)SR¹⁰ (R¹⁰ is as defined above).

Examples of the “optionally amidated carboxyl” for R⁵ include a group represented by —CONR⁸R⁹ (R⁸ and R⁹ is as defined above).

Examples of the “hydrocarbon group optionally having substituent(s)” for R⁷ include (1) alkyl optionally having substituent(s), (2) cycloalkyl optionally having substituent(s), (3) alkenyl optionally having substituent(s), (4) aryl optionally having substituent(s) and (5) aralkyl optionally having substituent(s).

Examples of the “alkyl optionally having substituent(s)”, “cycloalkyl optionally having substituent(s)”, “alkenyl optionally having substituent(s)”, “aryl optionally having substituent(s)” and “aralkyl optionally having substituent(s)” as the “hydrocarbon group optionally having substituent(s)” for R⁷ include those similar to the “alkyl optionally having substituent(s)”, “cycloalkyl optionally having substituent(s)”, “alkenyl optionally having substituent(s)”, “aryl optionally having substituent(s)” and “aralkyl optionally having substituent(s)” as the aforementioned “group via a carbon atom” for R¹¹, R¹² or R⁶.

As R¹¹, C₆₋₁₄ aryl optionally having substituent(s) is preferable.

As R¹², (1) C₁₋₆ alkyl having a group via a nitrogen atom (particularly C₁₋₃ alkyl) or (2) a group via a nitrogen atom is preferable.

As R¹³, a group represented by —(CH₂)ₚQ (p is an integer of 0 to 3, and Q is a homocyclic group optionally having substituent(s) or a heterocyclic group optionally having substituent(s)) is preferable.

As R¹⁴, (1) C₁₋₆ alkyl optionally having C₁₋₆ alkoxy or (2) C₆₋₁₄ aryl optionally having substituent(s) is preferable.

As R⁵, —C(O)R⁷ (R⁷ is a hydrocarbon group optionally having substituent(s)) is preferable.

As R⁶, a hydrogen atom is preferable.

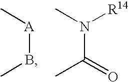

is preferable.

As compound (I), a compound represented by the formula

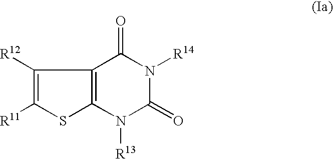

wherein each symbol is as defined above (hereinafter to be abbreviated as compound (Ia)) is preferable. Particularly, compound (Ia) wherein R¹¹ is C₆₋₁₄ aryl optionally having substituent(s), R¹² is (1) C₁₋₃ alkyl having a group via a nitrogen atom or (2) a group via a nitrogen atom, R¹³ is a group represented by —(CH₂)ₚQ (p is an integer of 0 to 3, and Q is a homocyclic group optionally having substituent(s) or a heterocyclic group optionally having substituent(s)), and R¹⁴ is (1) C₁₋₆ alkyl optionally having C₁₋₆ alkoxy or (2) C₆₋₁₄ aryl optionally having substituent(s) is preferable.

Specifically, in compound (I), a compound represented by the formula

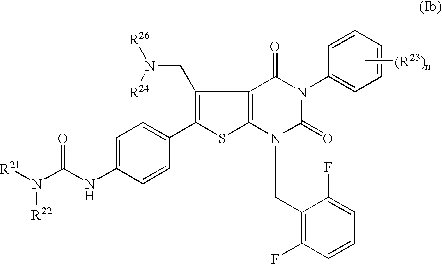

wherein R²¹ and R²² are each (1) a hydrogen atom, (2) hydroxy, (3) C₁₋₄ alkoxy, (4) C₁₋₄ alkoxy-carbonyl or (5) C₁₋₄ alkyl optionally having substituent(s),
R²³ is (1) a hydrogen atom, (2) a halogen atom, (3) hydroxy or (4) C₁₋₄ alkoxy optionally having substituent(s), or
the adjacent two R²³ are optionally bonded to form C₁₋₄ alkylenedioxy,
R²⁴ is (1) a hydrogen atom or (2) C₁₋₄ alkyl,
R²⁶ is (1) C₁₋₄ alkyl optionally having substituent(s) or (2) a group represented by the formula

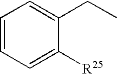

wherein R²⁵ is a hydrogen atom, or optionally bonded to R²⁴ to form heterocycle, and n is an integer of 0 to 5 (hereinafter to be abbreviated as compound (Ib)) is preferable.

Examples of the “C₁₋₄ alkoxy” for R²¹ or R²² include methoxy, ethoxy, propoxy, isopropoxy, butoxy, isobutoxy, sec-butoxy, tert-butoxy and the like. Of these, C₁₋₃ alkoxy is preferable and methoxy is more preferable.

Examples of the “C₁₋₄ alkoxy-carbonyl” for R²¹ or R²² include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl and the like. Of these, C₁₋₃ alkoxy-carbonyl is preferable and methoxycarbonyl is more preferable.

Examples of the “C₁₋₄ alkyl” of the “C₁₋₄ alkyl optionally having substituent(s)” for R²¹ or R²² include linear C₁₋₄ alkyl (e.g., methyl, ethyl, propyl, butyl and the like), branched C₃₋₄ alkyl (e.g., isopropyl, isobutyl, sec-butyl, tert-butyl and the like) and the like. Of these, C₁₋₃ alkyl is preferable and ethyl is more preferable.

Examples of the “substituent” of the “C₁₋₄ alkyl optionally having substituent(s)” for R²¹ or R²² include (i) hydroxy, (ii) C₁₋₇ acyloxy (e.g., C₁₋₆ alkyl-carbonyloxy such as acetoxy, propionyloxy and the like), (iii) benzoyloxy, (iv) amino optionally having 1 or 2 substituents selected from C₁₋₆ alkoxy-carbonyl (e.g., methoxycarbonyl, ethoxycarbonyl, tert-butoxycarbonyl and the like), benzyloxycarbonyl, C₁₋₄ acyl (e.g., C₁₋₃ alkyl-carbonyl such as acetyl, propionyl and the like, and the like), C₁₋₄ alkyl (e.g., methyl, ethyl, propyl, butyl and the like) and C₁₋₃ alkylsulfonyl (e.g., methanesulfonyl and the like) and the like (e.g., amino, dimethylamino, methoxycarbonylamino, ethoxycarbonylamino, tert-butoxycarbonylamino, benzyloxycarbonylamino, acetylamino, methanesulfonylamino and the like), (v) C₁₋₁₀ alkoxy (e.g., methoxy, ethoxy, propoxy, tert-butoxy and the like), (vi) C₃₋₇ cycloalkyloxycarbonyloxy-C₁₋₃ alkoxy (e.g., cyclohexyloxycarbonyloxy-1-ethoxy and the like), (vii) C₁₋₃ alkoxy-C₁₋₃ alkoxy (e.g., methoxymethoxy, methoxyethoxy and the like) and the like. Of these, hydroxy is preferable.

The “C₁₋₄ alkyl” of the “C₁₋₄ alkyl optionally having substituent(s)” for R²¹ or R²² may have 1 to 5, preferably 1 to 3, of the aforementioned substituents at substitutable position(s). When the number of the substituents is two or more, the substituents may be the same or different.

One of R²¹ and R²² is preferably a hydrogen atom and the other is preferably C₁₋₃ alkoxy.

Examples of the “halogen atom” for R²³ include fluorine, chlorine, bromine and iodine. Of these, chlorine is preferable.

Examples of the “C₁₋₄ alkoxy” of the “C₁₋₄ alkoxy optionally having substituent(s)” for R²³ include methoxy, ethoxy, propoxy, isopropoxy, butoxy, isobutoxy, sec-butoxy, tert-butoxy and the like. Of these, methoxy is preferable.

Examples of the “substituent” of the “C₁₋₄ alkoxy optionally having substituent(s)” for R²³ include those similar to the “substituent” of the aforementioned “C₁₋₄ alkyl optionally having substituent(s)” for R²¹ or R²². Of these, C₁₋₄ alkoxy is preferable.

The C₁₋₄ alkoxy may have 1 to 5, preferably 1 to 3, of the aforementioned substituents at substitutable position(s). When the number of the substituents is two or more, the substituents may be the same or different.

Examples of the “C₁₋₄ alkylenedioxy” formed by two adjacent R²³ bonded to each other include methylenedioxy, ethylenedioxy and the like.

R²³ is preferably a hydrogen atom.

Examples of the “C₁₋₄ alkyl” for R²⁴ include linear C₁₋₄ alkyl (e.g., methyl, ethyl, propyl, butyl and the like), branched C₃₋₄ alkyl (e.g., isopropyl, isobutyl, sec-butyl, tert-butyl and the like) and the like. Of these, C₁₋₃ alkyl is preferable and methyl is particularly preferable.

Examples of the “C₁₋₄ alkyl optionally having substituent(s)” for R²⁶ include those similar to the aforementioned “C₁₋₄ alkyl optionally having substituent(s)” for R²¹ or R²².

Examples of the “heterocycle” formed by R²⁴ and R²⁵ bonded to each other include a 5- or 6-membered nitrogen-containing heterocyclic group. When R²⁴ and R²⁵ are bonded, a group represented by the formula

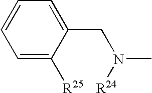

may be, for example, a group represented by the formula

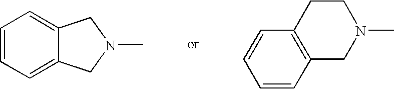

and the like. Of these, a group represented by the formula

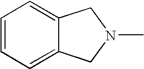

is preferable.

R²⁶ is preferably a group represented by the formula

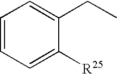

wherein R²⁵ is as defined above.

R²⁴ is preferably C₁₋₃ alkyl and R²⁵ is preferably a hydrogen atom.

n is preferably an integer of 0 to 2.

Of compounds (I), preferred are a compound represented by the formula

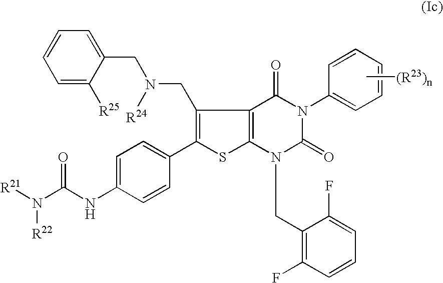

wherein each symbol is as defined above (hereinafter to be abbreviated as compound (Ic)) and the like.

More preferred are compound (Ic) wherein R²¹ is hydroxy, methoxy or C₁₋₃ alkyl;

R²² is a hydrogen atom or C₁₋₃ alkyl; R²⁴ is C₁₋₃ alkyl; R²⁵ is a hydrogen atom; and n is 0, and the like.

Particularly preferred are compound (Ic) wherein R²¹ is methoxy; R²² and R²⁵ are each a hydrogen atom; R²⁴ is C₁₋₃ alkyl; R²⁵ is a hydrogen atom; and n is 0, and the like.

Of compounds (I), also preferred is compound (Ib) wherein R²¹ is (i) hydroxy, (ii) C₁₋₄ alkoxy, or (iii) C₁₋₄ alkyl optionally having hydroxy or C₁₋₄ alkyl-carbonyloxy; R²² is a hydrogen atom, C₁₋₄ alkyl or C₁₋₄ alkoxy-carbonyl; R²³ is a hydrogen atom, a halogen atom, hydroxy or C₁₋₄ alkoxy-C₁₋₄ alkoxy, or two adjacent R²³ are bonded to form C₁₋₃ alkylenedioxy; R²⁴ is a hydrogen atom or C₁₋₃ alkyl; R²⁶ is C₁₋₄ alkoxy-C₁₋₄ alkyl or a group represented by the formula

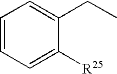

wherein R²⁵ is a hydrogen atom, or R²⁴ and R²⁵ are bonded to form a 5- or 6-membered heterocycle; and n is 1 or 2.

Specific examples of compound (I) include 5-(N-benzyl-N-methylaminomethyl)-1-(2,6-difluorobenzyl)-6-[4-(3-methoxyureido)phenyl]-3-phenylthieno[2,3-d]pyrimidine-2,4(1H,3H)-dione, 5-(N-benzyl-N-methylaminomethyl)-1-(2,6-difluorobenzyl)-6-[4-(3-hydroxyureido)phenyl]-3-phenylthieno[2,3-d]pyrimidine-2,4(1H,3H)-dione, 5-(N-benzyl-N-methylaminomethyl)-1-(2,6-difluorobenzyl)-6-[4-(3-methylureido)phenyl]-3-phenylthieno[2,3-d]pyrimidine-2,4(1H,3H)-dione, 5-(N-benzyl-N-methylaminomethyl)-1-(2,6-difluorobenzyl)-6-[4-(3-ethylureido)phenyl]-3-phenylthieno[2,3-d]pyrimidine-2,4(1H,3H)-dione and salts thereof.

Of these, 5-(N-benzyl-N-methylaminomethyl)-1-(2,6-difluorobenzyl)-6-[4-(3-methoxyureido)phenyl]-3-phenylthieno[2,3-d]pyrimidine-2,4(1H,3H)-dione (compound A) and a salt thereof are preferable.

Preferable examples of the nonpeptidic compound having a gonadotropin releasing hormone antagonistic action include a compound represented by the formula (I′)

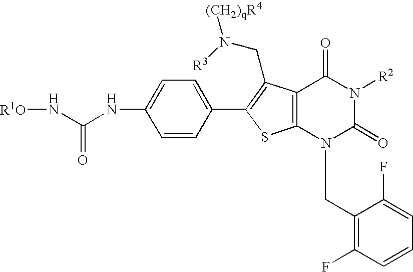

wherein R¹ is C₁₋₄ alkyl, R² is (1) C₁₋₆ alkyl optionally having substituent(s) selected from (1′) a hydroxyl group, (2′) C₁₋₄ alkoxy, (3′) C₁₋₄ alkoxy-carbonyl, (4′) di-C₁₋₄ alkyl-carbamoyl, (5′) a 5- to 7-membered nitrogen-containing heterocyclic group, (6′) C₁₋₄ alkyl-carbonyl and (7′) halogen, (2) C₃₋₈ cycloalkyl optionally having (1′) a hydroxyl group or (2′) mono-C₁₋₄ alkyl-carbonylamino, (3) a 5- to 7-membered nitrogen-containing heterocyclic group optionally having substituent(s) selected from (1′) halogen, (2′) a hydroxyl group, (3′) C₁₋₄ alkyl and (4′) C₁₋₄ alkoxy, (4) phenyl optionally having substituent(s) selected from (1′) halogen, (2′) C₁₋₄ alkoxy-C₁₋₄ alkyl, (3′) mono-C₁₋₄ alkyl-carbamoyl-C₁₋₄ alkyl, (4′) C₁₋₄ alkoxy and (5′) mono-C₁₋₄ alkylcarbamoyl-C₁₋₄ alkoxy or (5) C₁₋₄ alkoxy, R³ is C₁₋₄ alkyl, R⁴ is (1) a hydrogen atom, (2) C₁₋₄ alkoxy, (3) C₆₋₁₀ aryl, (4) N—C₁₋₄ alkyl-N—C₁₋₄ alkylsulfonylamino, (5) a hydroxyl group or (6) a 5- to 7-membered nitrogen-containing heterocyclic group optionally having substituent(s) selected from (1′) oxo, (2′) C₁₋₄ alkyl, (3′) hydroxy-C₁₋₄ alkyl, (4′) C₁₋₄ alkoxy-carbonyl, (5′) mono-C₁₋₄ alkyl-carbamoyl and (6′) C₁₋₄ alkylsulfonyl, and q is an integer of 1 to 4, provided when R² is phenyl optionally having substituent(s), R⁴ should be a 5- to 7-membered nitrogen-containing heterocyclic group optionally having substituent(s) selected from (1) oxo, (2) hydroxy-C₁₋₄ alkyl, (3) C₁₋₄ alkoxy-carbonyl, (4) mono-C₁₋₄ alkyl-carbamoyl and (5) C₁₋₄ alkylsulfonyl (hereinafter sometimes to be abbreviated as compound (I′)), a salt thereof and a prodrug thereof.

The definition of each substituent in the aforementioned compound (I′) is shown below.

Examples of the “C₁₋₄ alkyl” include linear C₁₋₄ alkyl (e.g., methyl, ethyl, propyl, butyl etc.), branched C₃₋₄ alkyl (e.g., isopropyl, isobutyl, sec-butyl, tert-butyl etc.) and the like.

Examples of the “C₁₋₆ alkyl” include linear C₁₋₆ alkyl (e.g., methyl, ethyl, propyl, butyl, pentyl, hexyl etc.), branched C₃₋₆ alkyl (e.g., isopropyl, isobutyl, sec-butyl, tert-butyl, isopentyl etc.) and the like.

Examples of the “C₁₋₄ alkoxy” include linear C₁₋₄ alkoxy (e.g., methoxy, ethoxy, propoxy, butoxy etc.), branched C₃₋₄ alkoxy (e.g., isopropoxy, isobutoxy, sec-butoxy, tert-butoxy etc.) and the like.

Examples of the “C₁₋₄ alkoxy-carbonyl” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl and the like.

Examples of the “di-C₁₋₄ alkyl-carbamoyl” include dimethylcarbamoyl, diethylcarbamoyl, dipropylcarbamoyl, diisopropylcarbamoyl, N-ethyl-N-methylcarbamoyl and the like.

Examples of the “5- to 7-membered nitrogen-containing heterocyclic group” include pyrrolidin-1-yl, pyrrolidin-2-yl, pyrrolidin-3-yl, oxazolidin-3-yl, thiazolidin-3-yl, isoxazolidin-2-yl, isothiazolidin-2-yl, imidazolidin-1-yl, imidazolidin-2-yl, imidazolidin-4-yl, pyrazolidin-1-yl, pyrazolidin-3-yl, pyrazolidin-4-yl, pyrrol-1-yl, pyrrol-2-yl, pyrrol-3-yl, imidazol-1-yl, imidazol-2-yl, imidazol-4-yl, pyrazol-1-yl, pyrazol-3-yl, pyrazol-4-yl, 1,2,3-triazol-1-yl, 1,2,5-triazol-1-yl, tetrazol-1-yl, tetrazol-2-yl, tetrazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, piperidin-1-yl, piperidin-2-yl, piperidin-3-yl, piperidin-4-yl, piperazin-1-yl, piperazin-2-yl, morpholin-2-yl, morpholin-3-yl, morpholin-4-yl, pyridin-2-yl, pyridin-3-yl, pyridin-4-yl, pyrazin-2-yl, pyrimidin-2-yl, pyrimidin-4-yl, pyrimidin-5-yl, pyridazin-3-yl, pyridazin-4-yl and the like. Of these, pyrrolidin-1-yl, pyrrolidin-2-yl, imidazol-1-yl, imidazol-2-yl, 1,2,3-triazol-1-yl, 1,2,5-triazol-1-yl, tetrazol-1-yl, tetrazol-2-yl, pyridin-2-yl, pyridin-4-yl and the like are preferable.

Examples of the “C₁₋₄ alkyl-carbonyl” include methyl-carbonyl, ethyl-carbonyl, propyl-carbonyl, isopropyl-carbonyl, butyl-carbonyl, isobutyl-carbonyl, sec-butyl-carbonyl, tert-butyl-carbonyl and the like.

Examples of the “halogen” include fluorine, chlorine, bromine and iodine.

Examples of the “mono-C₁₋₄ alkyl-carbonylamino” include methylcarbonylamino, ethylcarbonylamino, propylcarbonylamino, isopropylcarbonylamino, butylcarbonylamino, isobutylcarbonylamino, sec-butylcarbonylamino, tert-butylcarbonylamino and the like.

Examples of the “C₃₋₈ cycloalkyl” include cyclopropyl, cyclobutyl, cyclopentyl, cyclohexyl, cycloheptyl, cyclooctyl and the like.

Examples of the “C₁₋₄ alkoxy-C₁₋₄ alkyl” include methoxymethyl, 1-methoxyethyl, 2-methoxyethyl, 1-methoxypropyl, 2-methoxypropyl, 3-methoxypropyl, 1-methoxybutyl, 2-methoxybutyl, 3-methoxybutyl, 4-methoxybutyl, 1-methoxy-1-methylethyl, 2-methoxy-1-methylethyl, 1-methoxy-1-methylpropyl, 2-methoxy-1-methylpropyl, 3-methoxy-1-methylpropyl, 1-(methoxymethyl)propyl, 1-methoxy-2-methylpropyl, 2-methoxy-2-methylpropyl, 3-methoxy-2-methylpropyl, 2-methoxy-1,1-dimethylethyl, ethoxymethyl, 2-ethoxyethyl, 3-ethoxypropyl, 4-ethoxybutyl and the like.

Examples of the “mono-C₁₋₄ alkyl-carbamoyl-C₁₋₄ alkyl” include methylaminocarbonylmethyl, ethylaminocarbonylmethyl, 2-methylaminocarbonylethyl, 2-ethylaminocarbonylethyl and the like.

Examples of the “mono-C₁₋₄ alkyl-carbamoyl-C₁₋₄ alkoxy” include methylaminocarbonylmethoxy, ethylaminocarbonylmethoxy, 2-methylaminocarbonylethoxy, 2-ethylaminocarbonylethoxy and the like.

Examples of the “C₆₋₁₀ aryl” include phenyl, 1-naphthyl, 2-naphthyl and the like.

Examples of the “N—C₁₋₄ alkyl-N—C₁₋₄ alkylsulfonylamino” include N-methyl-N-methylsulfonylamino, N-ethyl-N-methylsulfonylamino, N-ethylsulfonyl-N-methylamino, N-ethyl-N-ethylsulfonylamino and the like.

Examples of the “hydroxy-C₁₋₄ alkyl” include hydroxymethyl, 1-hydroxyethyl, 2-hydroxyethyl, 1-hydroxypropyl, 2-hydroxypropyl, 3-hydroxypropyl, 1-hydroxybutyl, 2-hydroxybutyl, 3-hydroxybutyl, 4-hydroxybutyl, 1-hydroxy-1-methylethyl, 2-hydroxy-1-methylethyl, 1-hydroxy-1-methylpropyl, 2-hydroxy-1-methylpropyl, 3-hydroxy-1-methylpropyl, 1-(hydroxymethyl)propyl, 1-hydroxy-2-methylpropyl, 2-hydroxy-2-methylpropyl, 3-hydroxy-2-methylpropyl, 2-hydroxy-1,1-dimethylethyl and the like.

Examples of the “mono-C₁₋₄ alkyl-carbamoyl” include methylcarbamoyl, ethylcarbamoyl, propylcarbamoyl, isopropylcarbamoyl, butylcarbamoyl, isobutylcarbamoyl, sec-butylcarbamoyl, tert-butylcarbamoyl and the like.

Examples of the “C₁₋₄ alkylsulfonyl” include methylsulfonyl, ethylsulfonyl, propylsulfonyl, isopropylsulfonyl, butylsulfonyl, isobutylsulfonyl, sec-butylsulfonyl, tert-butylsulfonyl and the like.

R¹ is preferably methyl or ethyl, and particularly preferably methyl.

R² is preferably a 5- to 7-membered nitrogen-containing heterocyclic group optionally having substituent(s) selected from (1) halogen, (2) a hydroxyl group, (3) C₁₋₄ alkyl and (4) C₁₋₄ alkoxy. Particularly, pyridyl (pyridin-2-yl, pyridin-3-yl, pyridin-4-yl) optionally having substituent(s) selected from (1) halogen, (2) a hydroxyl group, (3) C₁₋₄ alkyl and (4) C₁₋₄ alkoxy, and particularly preferably unsubstituted pyridin-2-yl.

R³ is preferably methyl or ethyl, and particularly preferably methyl.

R⁴ is preferably C₁₋₄ alkoxy, and particularly preferably methoxy or ethoxy.

q is preferably 1 or 2, and particularly preferably 2.

A preferable example of the combination of R³, R⁴ and q is a combination of methyl for R³, a hydrogen atom for R⁴ and 1 for q.

Preferable examples of compound (I′) include N-(4-(1-(2,6-difluorobenzyl)-5-(((2-methoxyethyl)(methyl)amino)methyl)-2,4-dioxo-3-(2-pyridinyl)-1,2,3,4-tetrahydrothieno[2,3-d]pyrimidin-6-yl)phenyl)-N′-methoxyurea, N-(4-(1-(2,6-difluorobenzyl)-5-(((2-ethoxyethyl)(methyl)amino)methyl)-2,4-dioxo-3-(2-pyridinyl)-1,2,3,4-tetrahydrothieno[2,3-d]pyrimidin-6-yl)phenyl)-N′-methoxyurea, N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea and N-(4-(1-(2,6-difluorobenzyl)-5-((dimethylamino)methyl)-3-(6-methoxypyridin-3-yl)-2,4-dioxo-1,2,3,4-tetrahydrothieno[2,3-d]pyrimidin-6-yl)phenyl)-N′-methoxyurea.

As the salt of compounds (I) and (I′), physiologically acceptable acid addition salts are preferable. Examples of such salt include salts with inorganic acid (e.g., hydrochloric acid, hydrobromic acid, nitric acid, sulfuric acid, phosphoric acid and the like), salts with organic acid (e.g., formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like), and the like. When compounds (I) and (I′) have an acidic group, they may form physiologically acceptable salts with inorganic base (e.g., alkali metal salt such as sodium, potassium, calcium, magnesium and the like, alkaline earth metal, ammonia and the like) or organic base (e.g., trimethylamine, triethylamine, pyridine, picoline, ethanolamine, diethanolamine, triethanolamine, dicyclohexylamine, N,N′-dibenzylethylenediamine and the like).

Compound (I) can be produced by a known method, for example, the methods described in WO95/28405, JP-A-9-169766, WO96/24597, WO97/14697, WO97/41126, WO00/00493, WO00/56739, JP-A-2001-278884 and the like or a method according thereto.

Compound (I′) can be produced by a known method, for example, the methods described in WO04/067535 and the like or a method according thereto.

Compounds (I), (I′) and the below-mentioned compound (II) may be used as prodrugs. Prodrugs of the compound (I), (I′) and (II) (hereinafter to be also referred to as prodrug of compound (I) and the like) mean compounds which are converted to compound (I) and the like with a reaction due to an enzyme, an gastric acid, etc. under the physiological condition in the living body, that is, compounds which are converted to compound (I) and the like with oxidation, reduction, hydrolysis, etc. according to an enzyme; compounds which are converted to compound (I) and the like by hydrolysis etc. due to gastric acid, and the like.

A prodrug of compound (I) may be, when compound (I) and the like have amino, a compound obtained by subjecting the amino group to an acylation, alkylation or phosphorylation (e.g., a compound obtained by subjecting an amino group in compound (I) and the like to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation and tert-butylation, etc.); when compound (I) and the like have hydroxy, a compound obtained by subjecting the hydroxy group to an acylation, alkylation, phosphorylation or boration (e.g., a compound obtained by subjecting a hydroxy group in compound (I) and the like to an acetylation, palmitoylation, propanoylation, pivaloylation, succinylation, fumarylation, alanylation, dimethylaminomethylcarbonylation, etc.); when compound (I) and the like have carboxyl, a compound obtained by subjecting the carboxyl group to an esterification or amidation (e.g., a compound obtained by subjecting a carboxyl group in compound (I) and the like to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation, etc.) and the like. These compounds can be produced by a method known per se.

A prodrug for compound (I) and the like may also be one which is converted into compound (I) and the like under a physiological condition, such as those described in IYAKUHIN no KAIHATSU (Development of Pharmaceuticals), Vol. 7, Design of Molecules, p. 163-198, Published by HIROKAWA SHOTEN.

Prodrugs of compound (I) and the like may be as they are or pharmacologically acceptable salts. Examples of such salt include, when prodrugs of compound (I) and the like have an acidic group such as carboxyl and the like, salts with inorganic base (e.g., alkali metal such as sodium, potassium and the like, alkaline earth metal such as calcium, magnesium and the like, transition metal such as zinc, iron, copper and the like, etc.), organic base (e.g., organic amines such as trimethylamine, triethylamine, pyridine, picoline, ethanolamine, diethanolamine, triethanolamine, dicyclohexylamine, N,N′-dibenzylethylenediamine and the like, basic amino acids such as arginine, lysin, ornithine and the like, etc.) and the like.

When prodrugs of compound (I) and the like have a basic group such as amino and the like, examples thereof include salts with inorganic acid, organic acid (e.g., hydrochloric acid, nitric acid, sulfuric acid, phosphoric acid, carbonic acid, bicarbonic acid, formic acid, acetic acid, propionic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid etc.), acidic amino acid such as aspartic acid, glutamic acid and the like.

The prodrug of compound (I) and the like may be a hydrate or a non-hydrate.

When compounds (I), (I′) and the below-mentioned compound (II) have one or more asymmetric carbons in a molecule, both R-configuration and S-configuration due to such asymmetric carbon are also encompassed in the present invention.

Compounds (I), (I′) and the below-mentioned compound (II) may be labeled with an isotope (e.g., ³H, ¹⁴C, ³⁵S) and the like.

Examples of the nonpeptidic compound having a gonadotropin releasing hormone antagonistic action include a compound represented by the formula

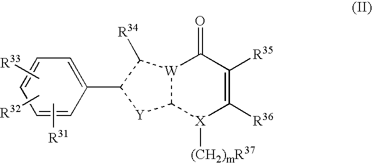

wherein one of W and Y is a nitrogen atom and the other is a carbon atom or both are nitrogen atoms, X is a nitrogen atom or a carbon atom, m is an integer of 0 to 3, R³¹, R³² and R³³ are the same or different and each is (i) a hydrogen atom or (ii) a group bonded via a carbon atom, a nitrogen atom, an oxygen atom or a sulfur atom, R³⁴ is a group bonded via a carbon atom, R³⁵ is a hydrogen atom, a halogen atom (e.g., fluorine, chlorine, bromine, iodine) or a group bonded via a carbon atom or an oxygen atom, R³⁶ is a hydrogen atom or a group bonded via a carbon atom, R³⁷ is a homocyclic group optionally having substituent(s) or a heterocyclic group optionally having substituent(s), and a broken line shows a single bond or a double bond (sometimes to be abbreviated as compound (II) in the present specification), a salt thereof and a prodrug thereof.

Each substituent in compound (II) is explained in detail in the following. In the aforementioned compound (II), examples of the group bonded via a carbon atom include (1) a hydrocarbon group optionally having substituent(s), (2) an acyl group optionally having substituent(s), (3) a heterocyclic group having a bond at a carbon atom, which optionally has substituent(s), (4) an optionally esterified or an amidated carboxyl group or (5) a cyano group.

In the aforementioned formula (II), examples of the group bonded via a nitrogen atom include (1) a nitro group or (2) a group represented by the formula —NR³⁸R³⁹ wherein R³⁸ is hydrogen, a hydrocarbon group optionally having substituent(s), an acyl group optionally having substituent(s), hydroxy optionally having substituent(s), a heterocyclic group optionally having substituent(s) or a group represented by the formula —S(O)ₜ—R⁴² wherein t is an integer of 0 to 2, and R⁴² is a hydrogen atom or a C₁₋₁₀ hydrocarbon group optionally having substituent(s), R³⁹ is hydrogen, a hydrocarbon group optionally having substituent(s) or an acyl group optionally having substituent(s), or R³⁸ and R³⁹ may be bonded to each other to form, together with the adjacent nitrogen atom, a cyclic amino group optionally having substituent(s).

In the aforementioned formula (II), examples of the group bonded via an oxygen atom include hydroxy optionally having substituent(s). The hydroxy optionally having substituent(s) is represented by, for example, the formula —OR⁴³ wherein R⁴³ is a hydrogen atom or a C₁₋₁₀ hydrocarbon group, a C₁₋₂₀ acyl group, a C₁₋₂₀ alkylsulfonyl group (e.g., methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl, hexylsulfonyl, heptylsulfonyl, octylsulfonyl, nonylsulfonyl, decylsulfonyl, undecylsulfonyl, dodecylsulfonyl, tridecylsulfonyl, tetradecylsulfonyl, pentadecylsulfonyl etc.), a C₆₋₁₄ arylsulfonyl group (e.g., phenylsulfonyl, 1-naphthylsulfonyl, 2-naphthylsulfonyl etc.) or a heterocyclic group, each of which optionally has substituent(s).

In the aforementioned formula (II), examples of the group bonded via a sulfur atom include a group represented by the formula —S(O)ᵣ—R⁴⁴ wherein r is an integer of 0 to 2, and R⁴⁴ is a hydrogen atom or a hydrocarbon group or a heterocyclic group, each of which optionally has substituent(s).

Examples of the aforementioned optionally esterified carboxyl group include a group represented by the formula —COOR⁵¹ wherein R⁵¹ is a hydrogen atom or a C₁₋₁₀ hydrocarbon group optionally having substituent(s).

Examples of the aforementioned optionally amidated carboxyl group include a group represented by the formula —CONR⁴⁵R⁴⁶ wherein R⁴⁵ is a hydrogen atom, or a hydrocarbon group or an alkoxy group (e.g., C₁₋₆ alkoxy such as methoxy, ethoxy, propoxy, isopropoxy, butoxy, isobutoxy, sec-butoxy, tert-butoxy, pentyloxy, hexyloxy and the like, etc.), each of which optionally has substituent(s). R⁴⁶ is a hydrogen atom or a hydrocarbon group optionally having substituent(s). R⁴⁵ and R⁴⁶ may form, together with the adjacent nitrogen atom, a cyclic amino group optionally having substituent(s). Examples of the optionally amidated carboxyl group include a group represented by —CONH₂, or a mono- or di-C₁₋₁₅ alkylcarbamoyl group, preferably a mono- or di-C₁₋₁₀ alkylcarbamoyl group (e.g., methylcarbamoyl, ethylcarbamoyl, propylcarbamoyl, isopropylcarbamoyl, butylcarbamoyl, isobutylcarbamoyl, sec-butylcarbamoyl, tert-butylcarbamoyl, pentylcarbamoyl, hexylcarbamoyl, dimethylcarbamoyl, methylethylcarbamoyl etc.) and the like.

Examples of the hydrocarbon group of the aforementioned hydrocarbon group optionally having substituent(s) include a C₁₋₂₀ hydrocarbon group (preferably, a C₁₋₁₀ hydrocarbon group) is preferable. Examples of the C₁₋₂₀ hydrocarbon group include (1) a C₁₋₁₅ alkyl group (e.g., methyl, ethyl, propyl, isopropyl, butyl, isobutyl, sec-butyl, tert-butyl, pentyl, hexyl, heptyl, octyl, nonyl, decyl, undecyl, dodecyl, tridecyl, tetradecyl, pentadecyl and the like. Of these, C₁₋₁₀ alkyl is preferable, and a C₁₋₆ alkyl group is particularly preferable), (2) a C₃₋₁₀ cycloalkyl group (e.g., cyclopropyl, cyclobutyl, cyclopentyl, cyclohexyl, cycloheptyl, cyclooctyl, cyclononyl and the like, with preference given to a C₃₋₆ cycloalkyl group), (3) a C₂₋₁₀ alkenyl group (e.g., vinyl, allyl, isopropenyl, 1-butenyl, 2-butenyl, 3-butenyl, butadienyl, 2-methylallyl, hexatrienyl, 3-octenyl and the like, with preference given to a C₂₋₆ alkenyl group), (4) a C₂₋₁₀ alkynyl group (e.g., ethynyl, 2-propynyl, butynyl, 3-hexynyl and the like, with preference given to a C₂₋₆ alkynyl group), (5) C₃₋₁₀ cycloalkenyl (e.g., cyclopropenyl, cyclopentenyl, cyclohexenyl and the like, with preference given to a C₃₋₆ cycloalkenyl group), (6) a C₆₋₁₄ aryl group (e.g., phenyl, naphthyl, anthryl, phenanthryl, acenaphthyl and the like, with preference given to phenyl and naphthyl) and (7) a C₇₋₂₀ aralkyl group (e.g., a C₆₋₁₄ aryl-C₁₋₆ alkyl group such as benzyl, phenethyl, benzhydryl and the like, with preference given to a phenyl-C₁₋₆ alkyl group such as benzyl, phenethyl and the like) and the like.

The aforementioned hydrocarbon group optionally has 1 to 6, preferably 1 to 5, more preferably 1 to 3, substituents at substitutable position(s). Examples of the substituent include (1) a halogen atom (e.g., fluorine, chlorine, bromine, iodine), (2) nitro, (3) nitroso, (4) cyano, (5) hydroxy optionally having a substituent [for example, (i) C₁₋₆ alkyl (said C₁₋₆ alkyl optionally has, as substituent(s), 1 to 3 from hydroxy, C₁₋₆ alkoxy, C₁₋₃ alkoxy-C₁₋₃ alkoxy, C₁₋₃ alkylthio, hydroxy-C₁₋₃ alkoxy, C₁₋₆ alkyl-carbonyl, carboxy, carbamoyl, C₁₋₆ alkyl-carbamoyl, a 5- to 8-membered heterocyclic group (similar to the below-mentioned “5- to 8-membered heterocyclic group containing, besides carbon atom(s), 1 to 4 hetero atoms selected from an oxygen atom, a sulfur atom, a nitrogen atom etc.”) and a halogen atom (e.g., fluorine, chlorine, bromine, iodine), (ii) C₁₋₄ acyl (e.g., C₁₋₄ alkanoyl (formyl, acetyl, propionyl, butyryl, isobutyryl etc.), C₃₋₄ alkenoyl (vinylcarbonyl, 1-propenylcarbonyl, 2-propenylcarbonyl etc.) etc.), (iii) C₇₋₂₀ aralkyl (said C₇₋₂₀ aralkyl group, namely, C₆₋₁₄ aryl-C₁₋₆ alkyl, optionally has, as substituent(s), 1 to 3, preferably 1, from a halogen atom (e.g., fluorine, chlorine, bromine, iodine), C₁₋₃ alkoxy and C₁₋₄ alkyl), (iv) C₆₋₁₄ aryl (said C₆₋₁₄ aryl optionally has, as substituent(s), 1 to 3, preferably 1, from a halogen atom (e.g., fluorine, chlorine, bromine, iodine)), (v) C₂₋₆ alkenyl, (vi) C₃₋₇ cycloalkyl, (vii) C₁₋₃ alkoxy-carbonyl, (viii) mono- or di-C₁₋₆ alkylamino, (ix) C₂₋₆ alkenylamino, (x) C₁₋₆ alkyl-carbonyl or (xi) C₃₋₆ cycloalkyloxy-carbonyl, (6) a group represented by the formula —S(O)ₛ—R⁴⁷ wherein s is an integer of 0 to 2, and R⁴⁷ is a hydrogen atom or a hydrocarbon group optionally having 1 to 3, preferably 1, substituent (e.g., a halogen atom (e.g., fluorine, chlorine, bromine, iodine), nitro, cyano, hydroxy, oxo, thioxo, carboxy, cyano-C₆₋₁₄ aryl, halogeno-C₆₋₁₄ aryl etc.) at any substitutable position(s), where the hydrocarbon group is preferably a C₁₋₂₀ hydrocarbon group, and particularly preferably C₁₋₆ alkyl, C₆₋₁₄ aryl or C₇₋₂₀ aralkyl], (7) an amino group optionally having substituent(s) [for example, a group represented by the formula —NR⁴⁸R⁴⁹ wherein R⁴⁸ and R⁴⁹ are the same or different and each is a hydrogen atom, C₁₋₆ alkyl, C₁₋₆ alkylamino-C₁₋₆ alkyl, C₁₋₆ alkoxy, C₂₋₆ alkenyl, C₃₋₇ cycloalkyl, phenyl, phenyl-C₁₋₆ alkyl, C₁₋₆ alkanoyl, C₃₋₆ alkenoyl, C₃₋₇ cycloalkyl-carbonyl, phenyl-C₁₋₆ alkyl-carbonyl, C₁₋₆ alkoxy-carbonyl, phenyl-C₁₋₆ alkoxy-carbonyl or a 5- to 8-membered heterocyclic group (similar to the below-mentioned “5- to 8-membered heterocyclic group containing, besides carbon atom(s), 1 to 4 hetero atoms selected from an oxygen atom, a sulfur atom, a nitrogen atom etc.”], (8) a group represented by the formula —COR⁵⁰ wherein R⁵⁰ is (i) a hydrogen atom, (ii) hydroxy, (iii) C₁₋₁₀ alkyl, (iv) C₁₋₆ alkoxy (said alkoxy is optionally substituted by C₆₋₁₄ aryl optionally having 1 to 3, preferably 1, substituent such as a halogen atom, nitro and the like at any substitutable position(s)), (v) C₃₋₆ cycloalkyl, (vi) C₆₋₁₄ aryl, (vii) C₆₋₁₄ aryloxy, (viii) C₇₋₂₀ aralkyl, (ix) an amino group optionally having substituent represented by the formula —NR⁴⁰R⁴¹ wherein. R⁴⁰ is hydrogen or a C₁₋₁₀ hydrocarbon group, a C₁₋₂₀ acyl group, hydroxy, a heterocyclic group or the formula —S(O)ₖ—R⁵² wherein k is an integer of 0 to 2, and R⁵² is a hydrogen atom, a C₁₋₁₀ hydrocarbon group optionally having substituent(s) or a heterocyclic group optionally having substituent(s), each of which optionally has substituent(s), and R⁴¹ is hydrogen or a C₁₋₁₀ hydrocarbon group, or R⁴⁰ and R⁴¹ may form, together with the adjacent nitrogen atom, a cyclic amino group optionally having substituent(s)), or (x) a 5- to 8-membered heterocyclic group (similar to the below-mentioned “5- to 8-membered heterocyclic group containing, besides carbon atom(s), 1 to 4 hetero atoms selected from an oxygen atom, a sulfur atom, a nitrogen atom etc.”)] (e.g., C₁₋₆ alkanoyl, C₃₋₆ alkenoyl, C₁₋₆ alkoxy-carbonyl and the like is preferable), (9) a 5- to 8-membered heterocyclic group containing 1 to 4 hetero atoms selected from a nitrogen atom, an oxygen atom and a sulfur atom, (10) sulfo, (11) C₆₋₁₄ aryl, (12) C₃₋₁₀ cycloalkyl, (13) C₁₋₆ alkylenedioxy (e.g., methylenedioxy, ethylenedioxy, propylenedioxy, 2,2-dimethylenedioxy etc.), (14) oxo; (15) thioxo, (16) C₂₋₄ alkynyl, (17) C₂₋₁₀ alkenyl (preferably, a C₂₋₆ alkenyl group), (18) C₇₋₂₀ aralkyl (e.g., C₆₋₁₄ aryl-C₁₋₆ alkyl), (19) amidino, (20) azido and the like.

Examples of the groups used for the explanation of the “substituent” of the aforementioned “hydrocarbon group” are shown in the following.

Examples of the C₁₋₁₀ alkyl include methyl, ethyl, propyl, isopropyl, butyl, isobutyl, sec-butyl, tert-butyl (i.e., C₁₋₄ alkyl), pentyl, hexyl (i.e., C₁₋₆ alkyl), heptyl, octyl, nonyl, decyl and the like.

Examples of the C₃₋₁₀ cycloalkyl include cyclopropyl, cyclobutyl, cyclopentyl, cyclohexyl (i.e., C₃₋₆ cycloalkyl), cycloheptyl (i.e., C₃₋₇ cycloalkyl), cyclooctyl, cyclononyl, cyclodecyl and the like.

Examples of the C₂₋₁₀ alkenyl include vinyl, allyl, isopropenyl, 1-butenyl, 2-butenyl, 3-butenyl, butadienyl, 2-methylallyl, hexatrienyl (i.e., C₂₋₆ alkenyl), 3-octenyl and the like.

Examples of the C₂₋₄ alkynyl include ethynyl, 2-propynyl, butynyl and the like.

Examples of the C₁₋₆ alkoxy include methoxy, ethoxy, propoxy, isopropoxy (i.e., C₁₋₃ alkoxy), butoxy, isobutoxy, sec-butoxy, tert-butoxy, pentyloxy, hexyloxy and the like.

Examples of the C₁₋₃ alkoxy-C₁₋₃ alkoxy include methoxymethoxy, methoxyethoxy, methoxypropoxy, ethoxymethoxy, ethoxyethoxy, ethoxypropoxy, propoxymethoxy, propoxyethoxy, propoxypropoxy and the like.

Examples of the C₁₋₃ alkylthio include methylthio, ethylthio, propylthio, isopropylthio and the like.

Examples of the hydroxy-C₁₋₃ alkoxy include hydroxymethoxy, 2-hydroxyethoxy, 3-hydroxypropoxy and the like.

Examples of the C₁₋₆ alkyl-carbonyl include acetyl, ethylcarbonyl, propylcarbonyl, butylcarbonyl, tert-butylcarbonyl, pentylcarbonyl, hexylcarbonyl and the like.

Examples of the C₃₋₇ cycloalkyl-carbonyl include cyclopropylcarbonyl, cyclobutylcarbonyl, cyclopentylcarbonyl, cyclohexylcarbonyl, cycloheptylcarbonyl and the like.

Examples of the C₁₋₆ alkoxy-carbonyl include methoxycarbonyl, ethoxycarbonyl, propoxycarbonyl, isopropoxycarbonyl (i.e., C₁₋₃ alkoxy-carbonyl), butoxycarbonyl, tert-butoxycarbonyl, pentyloxycarbonyl, hexyloxycarbonyl and the like.

Examples of the C₃₋₆ cycloalkyloxy-carbonyl include cyclopropyloxycarbonyl, cyclobutyloxycarbonyl, cyclopentyloxycarbonyl, cyclohexyloxycarbonyl and the like.

Examples of the phenyl-C₁₋₆ alkyl-carbonyl include benzylcarbonyl, phenethylcarbonyl and the like.

Examples of the phenyl-C₁₋₆ alkoxy-carbonyl include benzyloxycarbonyl, phenethyloxycarbonyl and the like.

Examples of the C₁₋₆ alkyl-carbamoyl include methylcarbamoyl, ethylcarbamoyl, propylcarbamoyl, isopropylcarbamoyl, butylcarbamoyl, isobutylcarbamoyl, sec-butylcarbamoyl, tert-butylcarbamoyl, pentylcarbamoyl, hexylcarbamoyl and the like.

Examples of the C₁₋₆ alkanoyl include formyl, acetyl, propionyl, butyryl, isobutyryl and the like.

Examples of the C₃₋₆ alkenoyl include vinylcarbonyl, 1-propenylcarbonyl, 2-propenylcarbonyl, 1-butenylcarbonyl, 1-pentenylcarbonyl and the like.

Examples of the C₆₋₁₄ aryl include phenyl, naphthyl, anthryl, phenanthryl, acenaphthyl and the like.

Examples of the cyano-C₆₋₁₄ aryl include 2-cyanophenyl, 3-cyanophenyl, 4-cyanophenyl and the like.

Examples of the halogeno-C₆₋₁₄ aryl include 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl 2,6-difluorophenyl, 2,3-dichlorophenyl, 2,4-dichlorophenyl, 2,5-dichlorophenyl, 2,6-dichlorophenyl and the like.

Examples of the C₇₋₂₀ aralkyl, namely, C₆₋₁₄ aryl-C₁₋₆ alkyl, include benzyl, phenethyl and the like.

Examples of the C₆₋₁₄ aryloxy include phenoxy, 1-naphthyloxy, 2-naphthyloxy and the like.

Examples of the mono- or di-C₁₋₆ alkylamino include methylamino, ethylamino, propylamino, isopropylamino, butylamino, dimethylamino, diethylamino and the like.

Examples of the C₂₋₆ alkenylamino include vinylamino, allylamino, isopropenylamino, 1-butenylamino, 2-butenylamino, 3-butenylamino, butadienylamino, 2-methylallylamino and the like.

Examples of the C₁₋₆ alkylamino-C₁₋₆ alkyl include methylaminomethyl, ethylaminomethyl, propylaminomethyl, methylaminoethyl, ethylaminoethyl and the like.

Examples of the phenyl-C₁₋₆ alkyl include benzyl, phenethyl and the like.

Of the aforementioned substituents on the hydrocarbon group, which have substituent(s), (9) a 5- to 8-membered heterocyclic group containing 1 to 4 hetero atoms selected from a nitrogen atom, an oxygen atom and a sulfur atom, (11) C₆₋₁₄ aryl, (12) C₃₋₁₀ cycloalkyl, (16) C₂₋₄ alkynyl, (17) C₂₋₁₀ alkenyl, (18) C₇₋₂₀ aralkyl and the like may further have 1 to 4, preferably 1 to 3, substituents at any substitutable position(s). Examples of the optional further substituent include 1 to 3 groups, more preferably 1 or 2 groups, selected from (1) hydroxy, (2) amino, (3) mono- or di-C₁₋₄ alkylamino (e.g., methylamino, ethylamino, propylamino, dimethylamino, diethylamino etc.), (4) C₁₋₄ alkoxy (e.g., methoxy, ethoxy, propoxy, isopropoxy, butoxy, isobutoxy, sec-butoxy, tert-butoxy etc.), (5) a halogen atom (e.g., fluorine, chlorine, bromine, iodine), (6) nitro and (7) C₁₋₆ alkyl (e.g., methyl, ethyl, propyl, isopropyl, butyl, isobutyl, sec-butyl, tert-butyl, pentyl, hexyl etc.) and the like.

When the hydrocarbon group is C₃₋₁₀ cycloalkyl, C₃₋₁₀-cycloalkenyl, C₆₋₁₄ aryl or C₇₋₂₀ aralkyl, it may have 1 to 3 substituents from C₁₋₆ alkyl (e.g., methyl, ethyl, propyl, isopropyl, butyl, isobutyl, sec-butyl, tert-butyl, pentyl, hexyl etc.), and said C₁₋₆ alkyl is optionally further substituted by 1 to 3 hydroxy, oxo, C₁₋₆ alkoxy (e.g., methoxy, ethoxy, propoxy, isopropoxy, butoxy, isobutoxy, sec-butoxy, tert-butoxy, pentyloxy, hexyloxy etc.), C₁₋₃ alkylthio (e.g., methylthio, ethylthio, propylthio, isopropylthio etc.), a halogen atom (e.g., fluorine, chlorine, bromine, iodine), carbamoyl and the like.

Examples of the substituted C₁₋₆ alkyl include formyl (methyl is substituted by oxo), carboxyl (methyl is substituted by oxo and hydroxy), C₁₋₆ alkoxycarbonyl (methyl is substituted, by oxo and alkoxy) (e.g., C₁₋₆ alkoxycarbonyl such as methoxycarbonyl, ethoxycarbonyl, tert-butoxycarbonyl and the like), hydroxy-C₁₋₆ alkyl (e.g., hydroxymethyl, hydroxyethyl, hydroxybutyl, hydroxypropyl etc.), C₁₋₃ alkoxy-C₁₋₆ alkyl (e.g., methoxymethyl, ethoxymethyl, ethoxybutyl, propoxymethyl, propoxyhexyl etc.) and the like.

While the number of the aforementioned substituents is 1 to 6, 1 to 5 is preferable, 1 to 3 is more preferable, and 1 or 2 is most preferable. The number of the substituents that the substituent optionally further has is preferably 1 to 4, particularly preferably 1 to 3, and most preferably 1 or 2.

Examples of the acyl group of the aforementioned acyl group optionally having substituent(s) recited as one example of the group bonded via a carbon atom, R³⁸ or R³⁹ include a C₁₋₂₀ acyl group. Examples thereof include formyl, C₁₋₆ alkyl-carbonyl (e.g., acetyl, ethylcarbonyl, propylcarbonyl, tert-butylcarbonyl etc.), C₁₋₆ alkoxy-carbonyl (e.g., methoxycarbonyl, ethoxycarbonyl, tert-butoxycarbonyl etc.), C₆₋₁₄ aryl-carbonyl (e.g., benzoyl, naphthoyl etc.), C₆₋₁₄ aryloxy-carbonyl (e.g., phenoxycarbonyl etc.), C₇₋₁₅ aralkyl-carbonyl (e.g., C₆₋₁₄ aryl-C₁₋₆ alkyl-carbonyl such as benzylcarbonyl and the like), C₇₋₁₉ aralkyloxy-carbonyl (e.g., C₆₋₁₄ aryl-C₁₋₆ alkoxy-carbonyl such as benzyloxycarbonyl and the like), C₂₋₄ alkenyl-carbonyl (e.g., 2-propenylcarbonyl etc.), C₃₋₆ cycloalkyl-carbonyl (e.g., cyclopropylcarbonyl etc.), tricyclic C₉₋₁₀ crosslinking cyclic hydrocarbon-carbonyl (e.g., adamantylcarbonyl etc.), heterocyclyl-carbonyl (e.g., (1) 5-membered heterocyclyl-carbonyl containing, besides carbon atom(s), 1 to 4 hetero atoms selected from an oxygen atom, a sulfur atom, a nitrogen atom and the like, such as thienylcarbonyl, furylcarbonyl, pyrrolylcarbonyl, pyrrolinylcarbonyl, oxazolylcarbonyl, thiazolylcarbonyl, pyrazolylcarbonyl, imidazolylcarbonyl, imidazolinylcarbonyl, isoxazolylcarbonyl, isothiazolylcarbonyl, 1,2,4-oxadiazolylcarbonyl, 1,3,4-oxadiazolylcarbonyl, furazanylcarbonyl, 1,2,4-thiadiazolylcarbonyl, 1,2,3-thiadiazolylcarbonyl, 1,2,5-thiadiazolylcarbonyl, 1,2,3-triazolylcarbonyl, 1,2,4-triazolylcarbonyl, triazinylcarbonyl, triazolidinylcarbonyl, 1H- or 2H-tetrazolylcarbonyl and the like; (2) 6-membered heterocyclyl-carbonyl containing, besides carbon atom(s), 1 to 4 hetero atoms selected from an oxygen atom, a sulfur atom, a nitrogen atom and the like, such as pyridylcarbonyl, pyrimidinylcarbonyl, thiomorpholinylcarbonyl, morpholinylcarbonyl, triazinylcarbonyl, pyrrolidinylcarbonyl, piperidinylcarbonyl, pyranylcarbonyl, thiopyranylcarbonyl, 1,4-oxazinylcarbonyl, 1,4-thiazinylcarbonyl, 1,3-thiazinylcarbonyl, piperazinylcarbonyl, triazinylcarbonyl, oxotriazinylcarbonyl, pyridazinylcarbonyl, pyrazinylcarbonyl and the like), carbamoyl, N—C₁₋₆ alkyl-carbamoyl (e.g., methylcarbamoyl, ethylcarbamoyl, propylcarbamoyl, isopropylcarbamoyl, butylcarbamoyl, isobutylcarbamoyl, tert-butylcarbamoyl, pentylcarbamoyl, hexylcarbamoyl etc.), N,N-di-C₁₋₆ alkyl-carbamoyl (e.g., dimethylcarbamoyl, diethylcarbamoyl, dipropylcarbamoyl, diisopropylcarbamoyl, dibutylcarbamoyl etc.) and the like.

Examples of the substituent of the acyl group optionally having substituent(s) include those similar to the substituent of the aforementioned hydrocarbon group optionally having substituent(s).

Examples of the heterocyclic group of the heterocyclic group or heterocyclic group optionally having substituent(s) in the aforementioned compound (II) include a 5- to 8-membered heterocyclic group containing, besides carbon atom(s), 1 to 4 hetero atoms selected from an oxygen atom, a sulfur atom, a nitrogen atom and the like, a dicyclic or tricyclic fused heterocyclic group formed by condensation of the same or different 2 or 3 of such heterocyclic groups, a dicyclic or tricyclic fused heterocycle group formed by condensation of the heterocyclic group and 1 or 2 benzene rings, and the like.

Specific examples of the heterocyclic group include (1) a 5-membered heterocyclic group containing, besides carbon atom(s), 1 to 4 hetero atoms selected from an oxygen atom, a sulfur atom, a nitrogen atom and the like, such as thienyl, furyl, pyrrolyl, pyrrolinyl, oxazolyl, thiazolyl, pyrazolyl, imidazolyl, imidazolinyl, isoxazolyl, isothiazolyl, 1,2,4-oxadiazolyl, 1,3,4-oxadiazolyl, furazanyl, 1,2,4-thiadiazolyl, 1,2,3-thiadiazolyl, 1,2,5-thiadiazolyl, 1,2,3-triazolyl, 1,2,4-triazolyl, triazinyl, triazolidinyl, 1H- or 2H-tetrazolyl and the like; (2) a 6-membered heterocyclic group containing, besides carbon atom(s), 1 to 4 hetero atoms selected from an oxygen atom, a sulfur atom, a nitrogen atom and the like such as pyridyl, pyrimidinyl, thiomorpholinyl, morpholinyl, triazinyl, pyrrolidinyl, piperidinyl, pyranyl, thiopyranyl, 1,4-oxazinyl, 1,4-thiazinyl, 1,3-thiazinyl, piperazinyl, triazinyl, oxotriazinyl, pyridazinyl, pyrazinyl and the like; (3) a dicyclic or tricyclic fused heterocycle group containing, besides carbon atom(s), 1 to 4 hetero atoms selected from an oxygen atom, a sulfur atom, a nitrogen atom and the like such as benzofuryl, benzothiazolyl, benzoxazolyl, tetrazolo[1,5-b]pyridazinyl, triazolo[4,5-b.]pyridazinyl, benzimidazolyl, quinolyl, isoquinolyl, cinnolinyl, phthalazinyl, quinazolinyl, quinoxalinyl, indolizinyl, indolyl, quinolizinyl, 1,8-naphthyridinyl, purinyl, pteridinyl, dibenzofuranyl, carbazolyl, acrydinyl, phenanthridinyl, chromanyl, benzoxazinyl, phenazinyl, phenothiazinyl, phenoxazinyl and the like; and the like.

Examples of the substituent that the heterocyclic group optionally has include (1) C₁₋₆ alkyl (e.g., methyl, ethyl, propyl, isopropyl, butyl, isobutyl, sec-butyl, tert-butyl, pentyl, hexyl etc.), (2) C₂₋₆ alkenyl (e.g., vinyl, allyl, isopropenyl, 1-butenyl, 2-butenyl, 3-butenyl, butadienyl, 2-methylallyl, hexatrienyl etc.), (3) C₂₋₆ alkynyl (e.g., ethynyl, 2-propynyl, butynyl, 3-hexynyl etc.), (4) C₃₋₆ cycloalkyl (e.g., cyclopropyl, cyclobutyl, cyclopentyl, cyclohexyl etc.), (5) C₅₋₇ cycloalkenyl (e.g., cyclopentenyl, cyclohexenyl, cycloheptenyl etc.), (6) C₇₋₁₁ aralkyl (e.g., C₆₋₁₀ aryl-C₁₋₅ alkyl, such as benzyl, phenethyl and the like, preferably benzyl), (7) C₆₋₁₄ aryl (e.g., phenyl, naphthyl, anthryl, phenanthryl, acenaphthyl, anthracenyl etc., preferably phenyl), (8) C₁₋₆ alkoxy (e.g., methoxy, ethoxy, propoxy, isopropoxy, butoxy, isobutoxy, sec-butoxy, tert-butoxy, pentyloxy, hexyloxy etc.), (9) C₆₋₁₄ aryloxy (e.g., phenoxy etc.), (10) C₁₋₆ alkanoyl (e.g., formyl, acetyl, propionyl, butyryl, isobutyryl etc.), (11) C₆₋₁₄ aryl-carbonyl (e.g., benzoyl etc.), (12) C₁₋₆ alkanoyloxy (e.g., formyloxy, acetyloxy, propionyloxy, butyryloxy, isobutyryloxy etc.), (13) C₆₋₁₄ aryl-carbonyloxy (e.g., benzoyloxy etc.), (14) carboxyl, (15) C₁₋₆ alkoxy-carbonyl (e.g., methoxycarbonyl, ethoxycarbonyl, propoxycarbonyl, isopropoxycarbonyl, butoxycarbonyl, isobutoxycarbonyl, tert-butoxycarbonyl etc.), (16) carbamoyl, (17) N-mono-C₁₋₄ alkylcarbamoyl (e.g., N-methylcarbamoyl, N-ethylcarbamoyl, N-propylcarbamoyl, N-isopropylcarbamoyl, N-butylcarbamoyl etc.), (18) N,N-di-C₁₋₄ alkylcarbamoyl (e.g., N,N-dimethylcarbamoyl, N,N-diethylcarbamoyl, N,N-dipropylcarbamoyl, N,N-dibutylcarbamoyl etc.), (19) 3- to 6-membered cyclic aminocarbonyl (e.g., 1-aziridinylcarbonyl, 1-azetidinylcarbonyl, 1-pyrrolidinylcarbonyl, 1-piperidinylcarbonyl, N-methylpiperazinylcarbonyl, morpholinocarbonyl etc.), (20) a halogen atom (e.g., fluorine, chlorine, bromine, iodine), (21) mono-, di- or tri-halogeno-C₁₋₄ alkyl (e.g., chloromethyl, dichloromethyl, trifluoromethyl, trifluoroethyl etc.), (22) oxo, (23) amidino, (24) imino, (25) amino, (26) mono- or di-C₁₋₄ alkylamino (e.g., methylamino, ethylamino, propylamino, isopropylamino, butylamino, dimethylamino, diethylamino, dipropylamino, diisopropylamino, dibutylamino etc.), (27) a 3- to 6-membered cyclic amino group optionally containing, besides carbon atom(s) and one nitrogen atom, 1 to 3 hetero atoms selected from an oxygen atom, a sulfur atom, a nitrogen atom and the like (e.g., aziridinyl, azetidinyl, pyrrolidinyl, pyrrolinyl, pyrrolyl, imidazolyl, pyrazolyl, imidazolidinyl, piperidino, morpholino, dihydropyridyl, N-methylpiperazinyl, N-ethylpiperazinyl etc.), (28) C₁₋₆ alkanoylamino (e.g., formamido, acetamido, trifluoroacetamido, propionylamido, butyrylamido, isobutyrylamido etc.), (29) benzamido, (30) carbamoylamino, (31) N—C₁₋₄ alkylcarbamoylamino (e.g., N-methylcarbamoylamino, N-ethylcarbamoylamino, N-propylcarbamoylamino, N-isopropylcarbamoylamino, N-butylcarbamoylamino etc.), (32) N,N-di-C₁₋₄ alkylcarbamoylamino (e.g., N,N-dimethylcarbamoylamino, N,N-diethylcarbamoylamino, N,N-dipropylcarbamoylamino, N,N-dibutylcarbamoylamino etc.), (33) C₁₋₃ alkylenedioxy (e.g., methylenedioxy, ethylenedioxy etc.), (34) —B(OH)₂, (35) hydroxy, (36) epoxy (—O—), (37) nitro, (38) cyano, (39) mercapto, (40) sulfo, (41) sulfino, (42) phosphono, (43) sulfamoyl, (44) C₁₋₆ alkylsulfamoyl (e.g., N-methylsulfamoyl, N-ethylsulfamoyl, N-propylsulfamoyl, N-isopropylsulfamoyl, N-butylsulfamoyl etc.), (45) di-C₁₋₆ alkylsulfamoyl (e.g., N,N-dimethylsulfamoyl, N,N-diethylsulfamoyl, N,N-dipropylsulfamoyl, N,N-dibutylsulfamoyl etc.), (46) C₁₋₆ alkylthio (e.g., methylthio, ethylthio, propylthio, isopropylthio, n-butylthio, sec-butylthio, tert-butylthio etc.), (47) phenylthio, (48) C₁₋₆ alkylsulfinyl (e.g., methylsulfinyl, ethylsulfinyl, propylsulfinyl, butylsulfinyl etc.), (49) phenylsulfinyl, (50) C₁₋₆ alkylsulfonyl (e.g., methylsulfonyl, ethylsulfonyl, propylsulfonyl, butylsulfonyl etc.) and (51) phenylsulfonyl and the like.

The number of the substituents that the heterocyclic group optionally has is 1 to 6, preferably 1 to 31 more preferably 1 or 2.

Examples of the heterocyclic group of the heterocyclic group optionally having substituent(s), which has a bond at a carbon atom, include a 5- to 8-membered heterocyclic group containing, besides carbon atom(s), 1 to 4 hetero atoms selected from an oxygen atom, a sulfur atom, a nitrogen atom and the like, a dicyclic or tricyclic fused heterocyclic group formed by condensation of the same or different 2 or 3 of such heterocyclic groups, a dicyclic or tricyclic fused heterocycle group formed by condensation of such heterocyclic group and 1 or 2 benzene rings, and the like, each of which has a bond at a carbon atom constituting the heterocycle.

Specific examples of the heterocyclic group having a bond at a carbon atom include (1) a 5-membered heterocyclic group containing, besides carbon atom(s), 1 to 4 hetero atoms selected from an oxygen atom, a sulfur atom, a nitrogen atom and the like, such as thienyl (e.g., 2- or 3-thienyl), furyl (e.g., 2- or 3-furyl), pyrrolyl (e.g., 2- or 3-pyrrolyl), oxazolyl (e.g., 2-, 4- or 5-oxazolyl), thiazolyl (e.g., 2-, 4- or 5-thiazolyl), pyrazolyl (e.g., 3-, 4- or 5-pyrazolyl), pyrrolidinyl (e.g., 2- or 3-pyrrolidinyl), imidazolyl (e.g., 2-, 4- or 5-imidazolyl), imidazolinyl (e.g., 2-imidazolinyl, 4-imidazolidinyl), isoxazolyl (e.g., 3-, 4- or 5-isoxazolyl), isothiazolyl (e.g., 3-, 4- or 5-isothiazolyl), oxadiazolyl [for example, 3- or 5-(1,2,4-oxadiazolyl), 2-, 5- or 6-(1,3,4-oxadiazolyl)], thiadiazolyl[for example, 3- or 5-(1,2,4-thiadiazolyl), 2- or 5-(1,3,4-thiadiazolyl), 4- or 5-(1,2,3-thiadiazolyl), 3- or 4-(1,2,5-thiadiazolyl)], triazolyl [for example, 2- or 5-(1,2,3-triazolyl), 3- or 5-(1,2,4-triazolyl)], tetrazolyl [for example, 5-(1H- or 2H-tetrazolyl)] and the like; (2) a 6-membered heterocyclic group containing, besides carbon atom(s), 1 to 4 hetero atoms selected from an oxygen atom, a sulfur atom, a nitrogen atom and the like, such as pyridyl (e.g., 2-, 3- or 4-pyridyl), pyrimidinyl (e.g., 2-, 4- or 5-pyrimidinyl), thiomorpholinyl (e.g., 2- or 3-thiomorpholinyl), morpholinyl (e.g., 2- or 3-morpholinyl), triazinyl (e.g., 3- or 6-triazinyl), piperidinyl (e.g., 2-, 3- or 4-piperidinyl), pyranyl (e.g., 2- or 3-pyranyl), thiopyranyl (e.g., 2- or 3-thiopyranyl), oxazinyl [for example, 2- or 3-(1,4-oxazinyl)], thiazinyl [for example, 2- or 3-(1,4-thiazinyl), 1- or 4-(1,3-thiazinyl)], piperazinyl (e.g., 2- or 3-piperazinyl), triazinyl (e.g., 3- or 6-triazinyl), pyridazinyl (e.g., 3- or 4-pyridazinyl), pyrazinyl (e.g., 2- or 3-pyrazinyl), pyridazinyl (e.g., 3- or 4-pyridazinyl) and the like; (3) a dicyclic or tricyclic fused heterocycle group containing, besides carbon atom(s), 1 to 4 hetero atoms selected from an oxygen atom, a sulfur atom, a nitrogen atom and the like, such as benzofuryl, benzothiazolyl, benzoxazolyl, tetrazolo[1,5-b]pyridazinyl, triazolo[4,5-b]pyridazinyl, benzimidazolyl, quinolyl, isoquinolyl, cinnolinyl, phthalazinyl, quinazolinyl, quinoxalinyl, indolizinyl, indolyl, quinolizinyl, 1,8-naphthyridinyl, purinyl, pteridinyl, dibenzofuranyl, carbazolyl, acrydinyl, phenanthridinyl, chromanyl, benzoxazinyl, phenazinyl, phenothiazinyl, phenoxazinyl and the like, each of which has a bond at a carbon atom; and the like.

Examples of the group that may be a substituent of the heterocyclic group having a bond at a carbon atom include those similar to the substituent of the aforementioned heterocyclic group optionally having substituent(s).

Examples of the cyclic amino group of the aforementioned cyclic amino group and cyclic amino group optionally having substituent(s) include a 5- to 7-membered nitrogen-containing cyclic group optionally further having one atom selected from an oxygen atom, a sulfur atom and a nitrogen atom. Examples thereof include pyrrolidinyl, pyrrolinyl, pyrrolyl, pyrazolidinyl, pyrazolinyl, pyrazolyl, imidazolidinyl, imidazolinyl, imidazolyl, 1,2,3-triazinyl, 1,2,3-triazolidinyl, 1,2,3-triazolyl, 1,2,3,4-tetrazolyl, piperidinyl, piperazinyl, azepinyl, hexamethyleneimino, oxazolidino, morpholino, thiazolidino and thiomorpholino. Of these, 5- or 6-membered ones are preferable. For example, pyrrolidinyl, pyrazolinyl, pyrazolyl, piperidinyl, piperazinyl, morpholino and thiomorpholino are preferable.

The cyclic amino group may have 1 to 3 substituents at any substitutable position(s). Examples of the substituent include (1) C₁₋₆ alkyl (e.g., methyl, ethyl, propyl, isopropyl, butyl, isobutyl, sec-butyl, tert-butyl, pentyl, hexyl etc.), (2) C₆₋₁₄ aryl (e.g., phenyl, naphthyl, anthryl, phenanthryl, acenaphthyl etc.), (3) C₇₋₁₀ aralkyl (phenyl-C₁₋₄ alkyl (e.g., benzyl, phenethyl etc.)), (4) benzhydryl, (5) C₁₋₆ alkyl-carbonyl (e.g., acetyl, propionyl etc.), (6) C₆₋₁₄ aryl-carbonyl (e.g., benzoyl etc.) and (7) C₁₋₆ alkoxy-carbonyl (e.g., methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, hexyloxycarbonyl etc.) and the like. Preferable substituent is C₁₋₆ alkyl, and more preferable substituent is C₁₋₃ alkyl.

Examples of the homocyclic group of the homocyclic group optionally having substituent(s) include an optionally condensed 3- to 7-membered carbon cyclic group such as a C₆₋₁₀ aryl group (e.g., phenyl, naphthyl and the like), a C₃₋₇ cycloalkyl group (e.g., cyclopropyl, cyclobutyl, cyclopentyl, cyclohexyl, cycloheptyl etc.), C₃₋₇ cycloalkenyl (e.g., cyclopropenyl, cyclobutenyl, cyclopentenyl, cyclohexenyl, cycloheptenyl etc.) and the like, and the like.

The homocyclic group optionally has, at any substitutable position(s), 1 to 6, preferably 1 to 3, more preferably 1 or 2, substituents. Examples of the substituent include (1) C₁₋₁₅ alkyl (e.g., methyl, ethyl, propyl, isopropyl, butyl, isobutyl, sec-butyl, tert-butyl, pentyl, hexyl, heptyl, octyl, nonyl, decyl, undecyl, dodecyl, tridecyl, tetradecyl, pentadecyl etc.) optionally substituted by 1 to 3, preferably 1 or 2 halogen atoms (e.g., fluorine, chlorine, bromine, iodine) (preferably, C₁₋₆ alkyl optionally substituted by halogen atom(s)), (2) C₃₋₁₀ cycloalkyl (e.g., cyclopropyl, cyclobutyl, cyclopentyl, cyclohexyl, cycloheptyl, cyclooctyl, cyclononyl, cyclodecyl etc.), (3) C₂₋₁₀ alkenyl (e.g., vinyl, allyl, isopropenyl, 1-butenyl, 2-butenyl, 3-butenyl, butadienyl, 2-methylallyl, hexatrienyl, 3-octenyl etc.), (4) C₂₋₁₀ alkynyl (e.g., ethynyl, 2-propynyl, butynyl, 3-hexynyl etc.), (5) C₃₋₁₀ cycloalkenyl (e.g., cyclopropenyl, cyclopentenyl, cyclohexenyl etc.), (6) C₆₋₁₀ aryl (e.g., phenyl, naphthyl etc.), (7) C₇₋₂₀ aralkyl (e.g., benzyl, phenethyl etc.), (8) nitro, (9) hydroxy, (10) mercapto, (11) oxo, (12) thioxo, (13) cyano, (14) carbamoyl, (15) carboxyl, (16) C₁₋₆ alkoxy-carbonyl (e.g., methoxycarbonyl, ethoxycarbonyl etc.), (17) sulfo, (18) a halogen atom (e.g., fluorine, chlorine, bromine, iodine), (19) C₁₋₆ alkoxy (e.g., methoxy, ethoxy, propoxy, isopropoxy, butoxy, isobutoxy, sec-butoxy, tert-butoxy, pentyloxy, hexyloxy etc.), (20) C₆₋₁₀ aryloxy (e.g., phenoxy etc.), (21) C₁₋₆ acyloxy (e.g., C₁₋₆ alkanoyloxy, such as acetoxy, propionyloxy and the like, etc.), (22) C₁₋₆ alkylthio (e.g., methylthio, ethylthio, propylthio, isopropylthio, butylthio, tert-butylthio etc.), (23) C₆₋₁₀ arylthio (e.g., phenylthio etc.), (24) C₁₋₆ alkylsulfinyl (e.g., methylsulfinyl, ethylsulfinyl etc.), (25) C₆₋₁₀ arylsulfinyl (e.g., phenylsulfinyl etc.), (26) C₁₋₆ alkylsulfonyl (e.g., methylsulfonyl, ethylsulfonyl etc.), (27) C₆₋₁₀ arylsulfonyl (e.g., phenylsulfonyl etc.), (28) amino, (29) C₁₋₆ acylamino (e.g., C₁₋₆ alkanoylamino, such as acetylamino, propionylamino and the like, etc.), (30) mono- or di-C₁₋₄ alkylamino (e.g., methylamino, ethylamino, propylamino, isopropylamino, butylamino, dimethylamino, diethylamino etc.), (31) C₃₋₈ cycloalkylamino (e.g., cyclopropylamino, cyclobutylamino, cyclopentylamino, cyclohexylamino etc.), (32) C₆₋₁₀ arylamino (e.g., anilino etc.), (33) C₁₋₆ alkanoyl (e.g., formyl, acetyl, hexanoyl etc.), (34) C₆₋₁₀ aryl-carbonyl (e.g., benzoyl etc.), and (35) a 5- or 6-membered heterocyclic group containing 1 to 4 hetero atoms selected from, besides carbon atom(s), oxygen, sulfur, nitrogen and the like [for example, thienyl (e.g., 2- or 3-thienyl), furyl (e.g., 2- or 3-furyl), pyrazolyl (e.g., 3-, 4- or 5-pyrazolyl), thiazolyl (e.g., 2-, 4- or 5-thiazolyl), isothiazolyl (e.g., 3-, 4- or 5-isothiazolyl), oxazolyl (e.g., 2-, 4- or 5-oxazolyl), isoxazolyl (e.g., 3-, 4- or 5-isoxazolyl), imidazolyl (e.g., 2-, 4- or 5-imidazolyl), triazolyl (e.g., 1,2,3- or 1,2,4-triazolyl), tetrazolyl (e.g., 1H or 2H-tetrazolyl), pyridyl (e.g., 2-, 3- or 4-pyridyl), pyrimidinyl (e.g., 2-, 4- or 5-pyrimidinyl), pyridazinyl (e.g., 3- or 4-pyridanizyl), quinolyl, isoquinolyl, indolyl etc.] and the like.

Examples of the hydroxy optionally having substituent(s) for R³⁸ or R⁴⁰ include a group represented by the aforementioned formula —OR⁴³ wherein R⁴³ is as defined above.

R³¹, R³² and R³³ in the aforementioned formula are the same or different and each is preferably (i) hydrogen or (ii) the aforementioned group bonded via a carbon atom, a nitrogen atom or an oxygen atom. Particularly preferably, R³¹ is a C₁₋₁₅ alkyl group, a C₃₋₁₀ cycloalkyl group, a C₂₋₁₀ alkenyl group, a C₂₋₁₀ alkynyl group, a C₃₋₁₀ cycloalkenyl group, a C₆₋₁₄ aryl group, a C₇₋₂₀ aralkyl group or a C₁₋₂₀ acyl group, each of which optionally has substituent(s), a nitro group, a group represented by the formula —NR⁴⁰R⁴¹ wherein R⁴⁰ and R⁴¹ are as defined above, or a group represented by the formula —OR^{43a} wherein R^{43a} is a hydrogen atom or a C₁₋₁₀ hydrocarbon group, a C₁₋₂₀ acyl group, a C₁₋₂₀ alkylsulfonyl group, a C₆₋₁₄ arylsulfonyl group or a 5- to 8-membered heterocyclic group (similar to the aforementioned “5- to 8-membered heterocyclic group containing, besides carbon atom(s), 1 to 4 hetero atoms selected from an oxygen atom, a sulfur atom, a nitrogen atom and the like”), each of which optionally has substituent(s), and one of R³² and R³³ is hydrogen and the other is the aforementioned group bonded via a carbon atom, a nitrogen atom or an oxygen atom (preferably both of R³² and R³³ are hydrogen).

R³¹ is preferably a C₁₋₁₀ alkyl group (preferably, a C₁₋₆ alkyl group) optionally substituted by 1 to 3, preferably 1 hydroxy, a nitro group, an amino group, the formula —NR^{40a}R^{41a} wherein R^{40a} is hydrogen, R^{41a} is C₁₋₆ alkyl-carbonyl optionally substituted by 1 to 3, preferably 1, hydroxy, C₁₋₆ alkylamino-carbonyl or C₆₋₁₄ arylamino-carbonyl, which is optionally substituted by 1 to 3, preferably 1, C₁₋₆ alkoxy (e.g., methoxy, ethoxy, propoxy, isopropoxy, butoxy, isobutoxy, sec-butoxy, tert-butoxy, pentyloxy, hexyloxy etc.), or the formula —OR^{43b} wherein R^{43b} is hydrogen, C₁₋₁₀ alkyl or C₃₋₁₀ cycloalkyl, which is optionally substituted by 1 to 3, preferably 1, hydroxy or C₁₋₆ alkyl-carbonyl, a C₁₋₆ alkylsulfonyl group or a C₆₋₁₀ arylsulfonyl group, which is optionally substituted by 1 to 3, preferably 1, hydroxy.

In the aforementioned formula, R³⁴ is preferably (1) a C₁₋₁₀ hydrocarbon group optionally having substituent(s), (2) a C₁₋₂₀ acyl group optionally having substituent(s), (3) a heterocyclic group optionally having substituent(s), which has a bond at a carbon atom, (4) an optionally esterified or amidated carboxyl group, or (5) a cyano group. Of these, R³⁴ is preferably a C₁₋₁₅ alkyl group, a C₃₋₁₀ cycloalkyl group, a C₂₋₁₀ alkenyl group, a C₂₋₁₀ alkynyl group, a C₃₋₁₀ cycloalkenyl group, a C₆₋₁₄ aryl group or a C₇₋₂₀ aralkyl group, each of which optionally has substituent(s). More preferably, it is a C₁₋₆ alkyl group optionally having substituent(s) (e.g., an aminoalkyl group optionally having substituent(s) and the like). Preferable examples of R³⁴ include a group represented by the formula —(CH₂)ᵤ—NR^{40b}R^{41b} wherein u is an integer of 1 to 3, R^{40b} is hydrogen, a C₁₋₁₀ hydrocarbon group optionally having substituent(s), a C₁₋₂₀ acyl group optionally having substituent(s), hydroxy optionally having substituent(s) (a group represented by the aforementioned formula —OR⁴³), a heterocyclic group optionally having substituent(s) or a group represented by the formula —S(O)ₜR⁴² wherein t is an integer of 0 to 2, and R⁴² is a hydrogen atom or a C₁₋₁₀ hydrocarbon group optionally having substituent(s), and R^{41b} is hydrogen or a C₁₋₁₀ hydrocarbon group, or R^{40b} and R^{41b} optionally form, together with the adjacent nitrogen atom, a cyclic amino group optionally having substituent(s). More preferably, R³⁴ is a halogen atom, hydroxy optionally substituted by a C₁₋₂₀ acyl group, or a C₁₋₃ alkyl group optionally substituted by an amino group optionally substituted by C₁₋₁₀ alkyl and/or C₆₋₁₄ aryl-C₁₋₁₀ alkyl. Particularly preferably, it is N—C₁₋₆ alkyl-N-benzylaminomethyl.

In the aforementioned formula, the halogen atom for R³⁵ is, for example, fluorine, chlorine, bromine or iodine.

R³⁵ is preferably hydrogen, a C₁₋₁₅ alkyl group optionally having substituent(s), a C₃₋₁₀ cycloalkyl group optionally having substituent(s), a C₂₋₁₀ alkenyl group optionally having substituent(s), a C₂₋₁₀ alkynyl group optionally having substituent(s), a C₃₋₁₀ cycloalkenyl group optionally having substituent(s), a C₆₋₁₄ aryl group optionally having substituent(s), a C₇₋₂₀ aralkyl group optionally having substituent(s), a C₁₋₂₀ acyl group optionally having substituent(s), an optionally esterified or amidated carboxyl group, or the formula —OR^{43c} wherein R^{43c} is a hydrogen atom or a C₁₋₁₅ alkyl group, a C₃₋₁₀ cycloalkyl group, a C₂₋₁₀ alkenyl group, a C₂₋₁₀ alkynyl group, a C₃₋₁₀ cycloalkenyl group, a C₆₋₁₄ aryl group, a C₇₋₂₀ aralkyl group, a C₁₋₂₀ acyl group, a C₁₋₂₀ alkylsulfonyl group, a C₆₋₁₄ arylsulfonyl group or a heterocyclic group, each of which optionally has substituent(s). Of these, R³⁵ is preferably hydrogen, a C₁₋₁₅ alkyl group optionally substituted by 1 to 3, preferably 1, C₆₋₁₄ aryl or C₁₋₆ alkoxy group, C₁₋₆ alkyl-carbonyl, C₁₋₆ alkoxy-carbonyl (e.g., methoxycarbonyl, ethoxycarbonyl, tert-butoxycarbonyl etc.), C₆₋₁₄ aryl-carbonyl (e.g., benzoyl etc.), C₆₋₁₄ aryloxy-carbonyl (e.g., phenoxycarbonyl etc.), C₇₋₁₅ aralkyl-carbonyl (e.g., benzylcarbonyl etc.), C₇₋₁₉ aralkyloxy-carbonyl (e.g., benzyloxycarbonyl etc.), N—C₁₋₁₀ alkyl-N—(C₁₋₁₀ alkoxy)amino-carbonyl (e.g., N-methyl-N-methoxyamino-carbonyl etc.), C₁₋₁₅ alkyloxy, a C₁₋₂₀ arylsulfonyl group, each of which is optionally substituted by 1 to 3, preferably 1, hydroxy and the like. More preferably, R³⁵ is (1) a C₁₋₆ alkoxy-carbonyl group, (2) a C₆₋₁₄ aryl group optionally substituted a halogen atom or C₁₋₆ alkoxy, or (3) a phenyl-C₁₋₃ alkyl group.

In the aforementioned the formula, R³⁶ is preferably hydrogen, or a C₁₋₁₅ alkyl group, a C₃₋₁₀ cycloalkyl group, a C₂₋₁₀ alkenyl group, a C₂₋₁₀ alkynyl group, a C₃₋₁₀ cycloalkenyl group, a C₆₋₁₄ aryl group or a C₇₋₂₀ aralkyl group, each of which optionally has substituent(s). Of these, R³⁶ is preferably hydrogen or a C₁₋₁₀ alkyl group, more preferably hydrogen or a C₁₋₆ alkyl group.

In the aforementioned the formula, R³⁷ is a homocyclic group or a heterocyclic group, each of which optionally has substituent(s), preferably a C₆₋₁₄ aryl group optionally having substituent(s). R³⁷ is more preferably a phenyl group optionally substituted by 1 to 3, preferably 1 or 2, halogen atoms or C₁₋₆ alkoxy groups. Particularly preferably, it is a phenyl group optionally substituted by 1 or 2 halogen atoms.

In compound (II), m is an integer of 0 to 3, preferably, m is an integer of 0 to 2, more preferably, m is 0 or 1.

In the aforementioned formula, u is an integer of 1 to 3, preferably, u is 1 or 2, more preferably, u is 1.

In compound (II), one of W and Y is a nitrogen atom and the other is a carbon atom or both are nitrogen atoms, and X is a nitrogen atom or a carbon atom. Accordingly, compound (II) is, for example, a compound represented by the formula

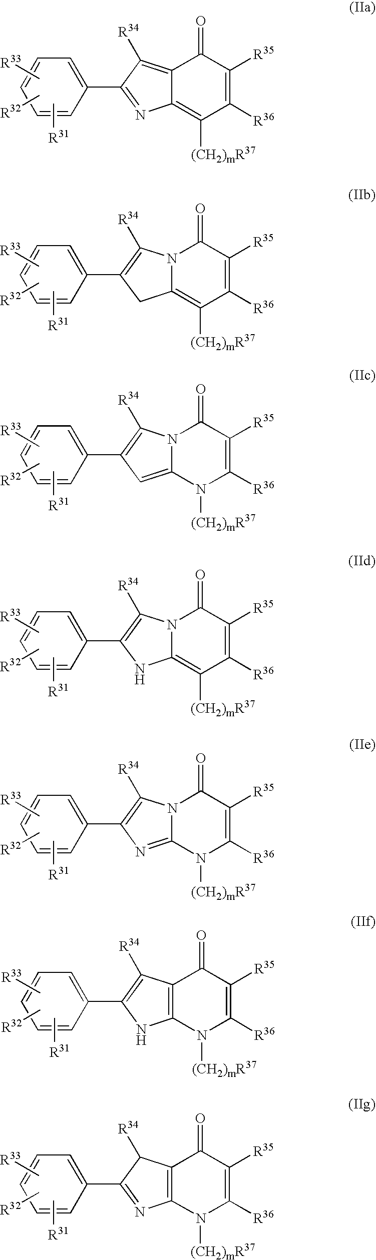

wherein each symbol is as defined above (preferably, a compound represented by the formula (IIa), (IIb), (IIc), (IId), (IIe) or (IIg)). Of these, compound (II) wherein X is a nitrogen atom is preferable, a compound represented by the formula (IIc) or (IIe) is particularly preferable, and a compound represented by the formula (IIe) is most preferable.

In compounds (II), a compound represented by the formula

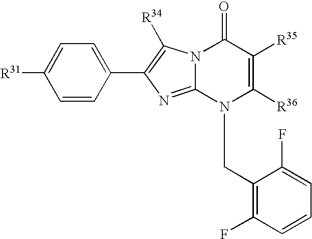

wherein each symbol is as defined above, is preferable. Particularly, a compound wherein R³¹ is (1) an amino group optionally substituted by (i) carbamoyl optionally substituted by C₁₋₆ alkyl or C₁₋₁₆ alkoxy, or (ii) C₁₋₆ alkyl-carbonyl or (2) a C₁₋₆ alkoxy group optionally substituted by C₃₋₆ cycloalkyl;
R³⁴ is an N—C₁₋₆ alkyl-N-benzylaminomethyl group;
R³⁵ is (1) a C₁₋₆ alkoxy-carbonyl group, (2) a C₆₋₁₄ aryl group optionally substituted by a halogen atom or C₁₋₆ alkoxy, or (3) a phenyl-C₁₋₃ alkyl group; and
R³⁶ is a hydrogen atom is more preferable.

In addition, a compound wherein R³¹ is (1) a nitro group, (2) an amino group optionally having 1 or 2 substituents selected from (i) C₁₋₆ alkyl optionally substituted by hydroxy, (ii) C₁₋₆ alkyl-carbonyl optionally substituted by hydroxy, halogen atom(s) or thienyl, (iii) C₆₋₁₀ aryl-carbonyl optionally substituted by C₁₋₆ alkyl, C₁₋₆ alkoxy or halogen atom(s), (iv) C₃₋₆ cycloalkyl-carbonyl, (v) C₂₋₄ alkenyl-carbonyl, (vi) C₁₋₆ alkoxy-carbonyl, (vii) C₁₋₆ alkylamino-carbonyl, (viii) C₁₋₆ alkoxyamino-carbonyl, (ix) phenylaminocarbonyl, (x) isoxazolylcarbonyl, thienylcarbonyl, thiazolylcarbonyl, pyrazolylcarbonyl or furylcarbonyl, each of which optionally has 1 or 2 substituents selected from C₁₋₆ alkyl, nitro and C₁₋₆ alkoxy, (xi) pyridylcarbonyl, (xii) C₁₋₆ alkylsulfonyl, (xiii) thienylsulfonyl and (xiv) phenylsulfonyl optionally substituted by C₁₋₆ alkyl, (3) a pyrrolyl group, or (4) a hydroxy group optionally substituted by C₁₋₆ alkyl, C₃₋₆ cycloalkyl-C₁₋₃ alkyl or C₁₋₆ alkyl-carbonyl; R³⁴ is a C₁₋₆ alkyl group optionally having 1 or 2 substituents selected from (1) a halogen atom, (2) hydroxy and (3) an amino optionally having 1 or 2 substituents selected from C₁₋₆ alkyl, phenyl-C₁₋₃ alkyl and di-C₁₋₆ alkylamino-C₁₋₃ alkyl; R³⁵ is (1) a halogen atom, (2) a phenyl group optionally substituted by a halogen atom or C₁₋₆ alkyl, or (3) a carbonyl group substituted by (i) C₁₋₆ alkyl, (ii) amino substituted by C₁₋₆ alkyl and C₁₋₆ alkoxy or (iii) C₁₋₆ alkoxy; and R³⁶ is a hydrogen atom or C₁₋₃ alkyl group is also preferable.

Specific examples of compound (II) include 8-(2,6-difluorobenzyl)-5,8-dihydro-2-[4-(ethylaminocarbonylamino)phenyl]-3-(N-methyl-N-benzylaminomethyl)-5-oxoimidazo[1,2-a]pyrimidine-6-carboxylic acid ethyl ester, 8-(2,6-difluorobenzyl)-5,8-dihydro-2-[4-(methoxyaminocarbonylamino)phenyl)]-3-(N-methyl-N-benzylaminomethyl)-5-oxoimidazo[1,2-a]pyrimidine-6-carboxylic acid isopropyl ester, 8-(2,6-difluorobenzyl)-5,8-dihydro-2-[4-(ethylaminocarbonylamino)phenyl]-3-(N-methyl-N-benzylaminomethyl)-5-oxoimidazo[1,2-a]pyrimidine-6-carboxylic acid isopropyl ester and the like.

Examples of the salt of compound (II) include those similar to the salt of the aforementioned compound (I).

Compound (II) can be produced by a known method, for example, the method described in WO99/33831, JP-A-11-315079 and the like or a method according thereto.

Moreover, examples of the nonpeptidic compound having a gonadotropin releasing hormone antagonistic action include quinoline derivatives described in WO97/14682 and JP-A-9-169735; imidazopyrimidine derivatives, pyrrolopyrimidine derivatives and triazolopyrimidine derivatives described in WO01/29044; imidazopyrimidine derivatives and pyrrolopyrimidine derivatives described in WO00/69859; compounds described in WO01/55119; compounds described in WO97/44037; compounds described in WO97/44041; compounds described in WO97/44321; compounds described in WO97/44339; 3,5,5,8,8-pentamethyl-5,6,7,8-tetrahydro-2-naphthalene derivatives described in Bioorganic & Medicinal Chemistry Letters 12 (2002) 3467-3470; 3,5,5,8,8-pentamethyl-5,6,7,8-tetrahydro-2-naphthalene derivatives and 5-[(3,5,5,8,8-pentamethyl-5,6,7,8-tetrahydro-2-naphthalenyl)methyl]-N-(2,4,6-trimethoxyphenyl)-2-furamide described in Bioorganic & Medicinal Chemistry Letters 12 (2002) 3635-3639; and the like.

When nonpeptidic compounds having a gonadotropin releasing hormone antagonistic action such as compounds (I), (I′) and (II) and the like have isomers such as tautomers, optical isomers, stereoisomers, positional isomers, rotational isomers and the like, any isomers and a mixture of isomers are also encompassed in the compounds of the present invention.

Furthermore, when nonpeptidic compounds having a gonadotropin releasing hormone antagonistic action such as compounds (I), (I′) and (II) and the like (hereinafter sometimes to be abbreviated as “the compound of the present invention”) have optical isomers, those resolved from the racemates are also encompassed in the compound of the present invention. These isomers can be respectively obtained as single products according to a synthesis method or separation method known per se (concentration, solvent extraction, column chromatography, recrystallization and the like).

The nonpeptidic compounds having a gonadotropin releasing hormone antagonistic action such as compounds (I), (I′) and (II) and the like may be crystals, and both single crystal form and crystal mixture are encompassed in the compound of the present invention. Crystals can be produced by crystallization according to a crystallization method known per se.

The nonpeptidic compounds having a gonadotropin releasing hormone antagonistic action such as compounds (I), (I′) and (II) and the like may be hydrates or non-hydrates. Examples of the hydrates include 1 hydrate, 1.5 hydrate, 2 hydrate and the like. When the compound of the present invention is obtained as a mixture of optically active forms, they can be resolved into the desired (R) form or (S) form according to an optical resolution method known per se. Moreover, the compound of the present invention may be labeled with an isotope (e.g., ³H, ¹⁴C, ³⁵S) and the like.

The nonpeptidic compound having a GnRH antagonistic action is low toxic and shows a superior GnRH antagonistic action and low toxicity. Moreover, it is superior in oral absorbability and action sustainability, as well as stability and pharmacokinetics. Furthermore, its production is easy and simple.

The “in vitro fertilization (IVF)” refers to a method comprising collecting an ovum, fertilizing the ovum in vitro with a spermatozoon and, when cleavage has progressed to a certain degree, inserting the ovum into the uterine cavity. That is, it includes the processes of ovulation induction, ovum collection, in vitro fertilization and culture, and embryo transfer.

The “embryo transfer” refers to, among the in vitro fertilization processes, the process of implanting embryo in the uterine cavity. One to several embryos inserted into the uterine cavity are implanted in the uterus, thereby resulting in pregnancy. The term also encompasses frozen embryo transfer and gamete intrafallopian transfer that do not involve in vitro fertilization.

The “in embryo transfer process” refers to the entire period during which insertion of an embryo or gamete into the uterine cavity or fallopian tube, a sequence of processes of implantation of the embryo or gamete in the uterus and pregnancy, drug administration before and after embryo transfer to achieve pregnancy, and the like are performed.

The “inhibition of premature ovulation” means inhibiting an ovulation from being ovulated earlier than the timing of ovum collection for in vitro fertilization, due to the ovulation induced by the natural period LH surge. Once natural ovulation occurs, exogenous collection of ovum becomes difficult, and in vitro fertilization cannot be performed.

The compound of the present invention suppresses secretion of gonadotropic hormone in mammals (e.g., human, monkey, bovine, horse, dog, cat, rabbit, rat, mouse and the like) by its GnRH receptor antagonistic action, and can be safely used for promoting and (or) assisting in vitro fertilization (IVF).

The compound of the present invention is used, for example, to collect an ovum in in vitro fertilization. For collecting a good ovum, an ovum is collected by exogenous control rather than natural ovulation, for which the compound of the present invention is used to eliminate an influence of endogenous LH. Specifically, while promoting ovum maturation with an ovulation inducing agent, premature ovulation is inhibited by simultaneous administration of the compound of the present invention. In addition, the compound of the present invention is used in embryo transfer process in in vitro fertilization, frozen embryo transfer and gamete intrafallopian transfer. By eliminating the influence of endogenous LH, from a stage prior to embryo or gamete implantation up to pregnancy through embryo implantation in the uterus after transfer, the uterus can be controlled to a condition suitable for achieving pregnancy.

Since the compound of the present invention is superior in oral absorbability and permits oral administration, it is superior as a promoter and (or) assistant agent for in vitro fertilization or a premature ovulation inhibitor used under controlled ovarian stimulation, as compared to the conventional peptidic GnRH antagonists administered by subcutaneous injection.

Therefore, the compound of the present invention is useful as a premature ovulation inhibitor for use in in vitro fertilization or embryo transfer process, an inhibitor of ovulation induced by endogenous LH in in vitro fertilization, an inhibitor of premature ovulation under controlled ovarian stimulation, and the like. Using the compound of the present invention, not only a mature ovum can be obtained certainly, but also the probability or stability of the fertilization of collected ovum, implantation in the uterus, achievement of pregnancy and maintenance of pregnancy can be expected.

The compound of the present invention can be used in combination in the process up to ovum collection in in vitro fertilization, when an ovulation inducing agent such as follicle-stimulating hormone, luteinizing hormone and the like is used to prompt ovum maturation and the like. In addition, the compound of the present invention can be used in embryo transfer process in combination with a promoter for implantation or pregnancy.

The compound of the present invention can be used in combination with gonadotropin (FSH, LH) or a pharmaceutical agent having a gonadotropin-like action; a GnRH superactive agonist such as Leuprorelin acetate, Gonadrelin, Buserelin, Triptorelin, Goserelin, Nafarelin, Histrelin, Deslorelin, Meterelin, Lecirelin and the like (preferably Leuprorelin acetate); or a GnRH antagonist such as Cetrorelix, Ganirelix, Abarelix, Nal-Blu, Antide, AzalineB, Degarelix, D63153, Teverelix and the like.

In addition, the compound of the present invention can also be used in combination with at least one kind of a steroidal or non-steroidal antiandrogen or antiestrogen, a chemotherapeutic agent, a peptidic GnRH antagonist, an α-reductase inhibitor, an α-receptor inhibitor, an aromatase inhibitor, a 17β-hydroxysteroid dehydrogenase inhibitor, an adrenal androgen production inhibitor, a kinase inhibitor, a hormonal therapeutic agent, growth factors or a pharmaceutical agent inhibiting the action of a receptor thereof and the like (hereinafter these are to be also referred to as concomitant drugs).

Examples of the “chemotherapeutic agent” include Ifosfamide, UTF, Adriamycin, Peplomycin, Cisplatin, Cyclophosphamide, 5-FU, UFT, Methotrexate, Mitomycin C, Mitoxantrone and the like.

Examples of the “adrenal androgen production inhibitor” include a lyase (C_{17,20}-lyase) inhibitor and the like.

Examples of the “kinase inhibitor” include tyrosine kinase and the like.

Examples of the “hormonal therapeutic agent” include antiestrogen, progestogen (e.g., MPA and the like), androgen, estrogen, anti-androgen and the like.

The “growth factors” may be any substances as long as it enhances cell growth, and generally, a factor which is a peptide having a molecular weight of not more than 20,000 and exhibits an action at a low concentration on binding with a receptor. Specific examples include (1) EGF (epidermal growth factor) or substances having an activity substantially identical to the activity thereof (e.g., EGF, heregulin (HER2 ligand) and the like), (2) insulin or substances having an activity substantially identical to the activity thereof (e.g., insulin, IGF (insulin-like growth factor)-1, IGF-2 and the like), (3) FGF (fibroblast growth factor) or substances having an activity substantially identical to the activity thereof (e.g., aFGF, bFGF, KGF (Keratindcyte Growth Factor), HGF (Hepatocyte Growth Factor), FGF-10 and the like), (4) growth factors (e.g., CSF (colony stimulating factor), EPO (erythropoietin), IL-2 (interleukin-2), NGF (nerve growth factor), PDGF (platelet-derived growth factor), TGFβ (transforming growth factorβ) and the like), and the like. The “receptor of the growth factors” may be any receptor capable of binding with the above-mentioned growth factors. Specific examples thereof include EGF receptor, heregulin receptor (HER2), insulin receptor-1, insulin receptor-2, IGF receptor, FGF receptor-1, FGF receptor-2 and the like. Examples of the pharmaceutical agent that inhibits the action of the above-mentioned growth factors include herceptin (HER2 receptor antibody) and the like. Examples of the pharmaceutical agent that inhibits the action of the above-mentioned growth factors or a receptor thereof include herbimycin, PD153035 (Science 265 (5175) p 1093, (1994)) and the like.

Moreover, HER2 inhibitors are also examples of the pharmaceutical agent that inhibits the action of the growth factors or a receptor thereof. The HER2 inhibitor may be any of antibodies, low-molecular-weight compounds (synthetic compounds, naturally occurring substances), antisenses, HER2 ligands, heregulin and those having a partially modified or altered structure, as long as it inhibits the activity of HER2 (e.g., phosphorylation activity). Furthermore, it may be a substance that inhibits the HER2 activity by inhibiting HER2 receptor (e.g., HER2 receptor antibody). Examples of the low-molecular-weight compound that has an HER2 inhibitory activity include compounds described in WO98/03505, specifically 1-[3-[4-[2-((E)-2-phenylethenyl)-4-oxazolylmethoxy]phenyl]-propyl]-1,2,4-triazole, and the like.

For breast cancer patients, the compound of the present invention may be used in combination with a pharmaceutical agent such as GnRH superactive agonist, antiestrogen, chemotherapeutic agent [e.g., Cyclophosphamide, 5-FU, UFT, Methotrexate, Adriamycin, Mitomycin C, Mitoxantrone and the like], peptidic GnRH antagonist, aromatase inhibitor, adrenal androgen production inhibitor, kinase inhibitor, hormonal therapeutic agent [e.g., antiestrogen (e.g., Tamoxifen and the like), progestogen (e.g., MPA and the like), androgen, estrogen and the like], a pharmaceutical agent that inhibits the action of growth factors or receptors thereof, and the like.

By combining the compound of the present invention and a concomitant drug, a superior effect such as

(1) the dose can be reduced as compared to single administration of the compound of the present invention or a concomitant drug,
(2) the drug to be used in combination with the compound of the present invention can be selected according to the condition of patients (mild case, severe case and the like),
(3) the period for inhibiting premature ovulation can be set longer by selecting a concomitant drug having different action and mechanism from those of the compound of the present invention,
(4) a sustained effect of premature ovulation inhibition can be designed by selecting a concomitant drug having different action and mechanism from those of the compound of the present invention,
(5) a synergistic effect can be afforded by a combined use of the compound of the present invention and a concomitant drug,
(6) side effects can be reduced by the combined use of the compound of the present invention and a concomitant drug,
(7) the compound of the present invention can also be administered to breast cancer patients and the like by the combined use of a concomitant drug, and the like, can be achieved.

In the following, a combined use of the compound of the present invention with a concomitant drug is referred to as “the combination agent of the present invention”.

When the compound of the present invention and the aforementioned concomitant drug is used in combination, the dose can be appropriately determined with the recommended minimum clinical dose of individual drugs as the standard, and in consideration of the age and body weight of the administration subject, symptom, administration time, administration method, dosage form, combination of drugs and the like. The dose of a particular patient is determined according to the age, body weight, general health status, sex, diet, administration time, administration method, clearance rate, drug combination, severity of the disease for which the patient is undergoing treatments, and other factors.

Typically, the respective daily dose of the compound of the present invention and at least one compound selected from various concomitant drugs or a salt thereof when used in combination is from not less than about 1/50 of the minimum recommended clinical dose to not more than the maximum recommended level of actual single administration thereof.

When using the combination agent of the present invention, the administration time of the compound of the present invention and the concomitant drug is not restricted, and the compound of the present invention or a pharmaceutical composition thereof and the concomitant drug or a pharmaceutical composition thereof can be administered to an administration subject simultaneously, or may be administered in a staggered manner. The dosage of the concomitant drug may be determined according to the administration amount clinically used, and can be appropriately selected depending on an administration subject, administration route, disease, combination and the like.

The administration mode of the concomitant drug is not particularly limited, and the compound of the present invention and the concomitant drug only need to be combined on administration. Examples of such administration mode include the following:

(1) administration of a single preparation obtained by simultaneously formulating the compound of the present invention and the concomitant drug, (2) simultaneous administration of two kinds of preparations of the compound of the present invention and the concomitant drug, which have been separately formulated, by the same administration route, (3) administration of two kinds of preparations of the compound of the present invention and the concomitant drug, which have been separately formulated, by the same administration route in a staggered manner, (4) simultaneous administration of two kinds of preparations of the compound of the present invention and the concomitant drug, which have been separately formulated, by different administration routes, (5) administration of two kinds of preparations of the compound of the present invention and the concomitant drug, which have been separately formulated, by different administration routes in a staggered manner (for example, administration in the order of the compound of the present invention and the concomitant drug, or in the reverse order) and the like.

When the compound of the present invention is used as the above-mentioned promoter or pharmaceutical aid (a premature ovulation inhibitor for use in in vitro fertilization or embryo transfer process), both oral administration and parenteral administration are available according to a method known per se. The compound is mixed with a pharmaceutically acceptable carrier, and generally administered orally in the solid dosage form of tablet, capsule, granule, powder and the like. Alternatively, it is parenterally administered intravenously, subcutaneously, intramuscularly and the like in the form of injection, suppository, sublingual tablet and the like. In addition, the compound may be administered sublingually, subcutaneously, intramuscularly and the like as a sustained release preparation such as sublingual tablet, microcapsule and the like.

While the daily dose of the compound of the present invention varies depending on the level of symptoms; age, sex, body weight and sensitivity difference of the subject of administration; timing of administration, interval, properties, dispensing and kind of pharmaceutical preparation; kind of the active ingredient, and the like and is not particularly limited, when used for the inhibition of premature ovulation in in vitro fertilization, the compound is generally administered in a dose of about 0.01-30 mg, preferably about 0.02-10 mg, more preferably 0.1-10 mg, most preferably 0.1-5 mg, per 1 kg body weight of a mammal, generally in 1-4 portions a day. When used for the inhibition of premature ovulation in embryo transfer process, the compound is generally administered in a dose of about 0.01-30 mg, preferably about 0.02-10 mg, more preferably 0.1-10 mg, most preferably 0.1-5 mg, per 1 kg body weight of a mammal, generally in 1-4 portions a day. While the dose for use in the animal husbandry or marine industry field is in accordance with the above-mentioned doses, the compound is generally administered in a dose of about 0.01-30 mg, preferably about 0.1-10 mg, per 1 kg body weight of an administration subject organism, generally in 1-3 portions a day. The content of compound (I) in the pharmaceutical composition of the present invention is about 0.01 to 100 wt % of the whole composition.

As the above-mentioned pharmaceutically acceptable carrier, various organic or inorganic carrier substances conventionally used as preparation materials can be used, and admixed as excipient, lubricant, binder and disintegrant for solid preparations; or solvent, solubilizing agent, suspending agent, isotonicity agent, buffer, soothing agent and the like for liquid preparations. Where necessary, preparation additives such as preservative, antioxidant, coloring agent, sweetening agent and the like can be used. Preferable examples of the above-mentioned excipient include lactose, sucrose, D-mannitol, starch, crystalline cellulose, light anhydrous silicic acid and the like. Preferable examples of the above-mentioned lubricant include magnesium stearate, calcium stearate, talc, colloidal silica and the like. Preferable examples of the above-mentioned binder include crystalline cellulose, sucrose, D-mannitol, dextrin, hydroxypropylcellulose, hydroxypropylmethylcellulose, polyvinylpyrrolidone and the like. Preferable examples of the above-mentioned disintegrant include starch, carboxymethylcellulose, calcium carboxymethylcellulose, croscarmellose sodium, carboxymethyl starch sodium and the like. Preferable examples of the above-mentioned solvent include water for injection, alcohol, propylene glycol, macrogol, sesame oil, corn oil and the like. Preferable examples of the above-mentioned solubilizing agents include polyethylene glycol, propylene glycol, D-mannitol, benzyl benzoate, ethanol, trisaminomethane, cholesterol, triethanolamine, sodium carbonate, sodium citrate and the like. Preferable examples of the above-mentioned suspending agent include surfactants such as stearyltriethanolamine, sodium lauryl sulfate, lauryl aminopropionic acid, lecithin, benzalkonium chloride, benzethonium chloride, glycerol monostearate and the like; hydrophilic polymers such as polyvinyl alcohol, polyvinylpyrrolidone, sodium carboxymethylcellulose, methylcellulose, hydroxymethylcellulose, hydroxyethylcellulose, hydroxypropylcellulose and the like; and the like. Preferable examples of the above-mentioned isotonicity agent include sodium chloride, glycerol, D-mannitol and the like. Preferable examples of the above-mentioned buffer include phosphate buffer, acetate buffer, carbonate buffer, citrate buffer and the like, and the like. Preferable examples of the soothing agent include benzyl alcohol and the like. Preferable examples of the above-mentioned preservative include paraoxybenzoates, chlorobutanol, benzyl alcohol, phenethyl alcohol, dehydroacetic acid, sorbic acid and the like. Preferable examples of the above-mentioned antioxidant include sulfite, ascorbic acid and the like.

The compound of the present invention can be formed into an intravenous, subcutaneous or intramuscular injection by adding a suspending agent, a solubilizing agent, a stabilizer, a tonicity agent, a preservative and the like and according to a method known per se. In this case, a freeze-dried product may be produced as necessary according to a method known per se. When the compound of the present invention is administered to, for example, a human, the compound can be safely administered orally or parenterally as it is or in the form of a pharmaceutical composition produced by admixing with an appropriate pharmacologically acceptable carrier, excipient or diluent. Examples of the above-mentioned pharmaceutical composition include oral preparations (e.g., powder, granule, capsule, tablet), and parenteral agents [e.g., injection, drip infusion, external preparation (e.g., preparation for nasal administration, dermal preparation and the like), suppositories (e.g., rectal suppository, vaginal suppository and the like), and the like]. These preparations can be produced according to a method known per se generally used for preparation forming steps.

The compound of the present invention can be formulated into an injection such as an aqueous injection in combination with a dispersing agent (e.g., Tween 80 (manufactured by Atlas Powder, US), HCO60 (manufactured by Nikko Chemicals) polyethylene glycol, carboxymethylcellulose, sodium alginate and the like), a preservative (e.g., methylparaben, propylparaben, benzyl alcohol and the like), an isotonicity agent (e.g., sodium chloride, mannitol, sorbitol, glucose and the like) and the like, or an oily injection by dissolving, suspending or emulsifying in a vegetable oil such as olive oil, sesame oil, cottonseed oil, corn oil and the like, or propylene glycol and the like. To formulate an oral preparation, the compound of the present invention is mixed with, for example, an excipient (e.g., lactose, sucrose, starch and the like), a disintegrant (e.g., starch, calcium carbonate and the like), a binder (e.g., starch, gum arabic, carboxymethylcellulose, polyvinylpyrrolidone, hydroxypropylcellulose and the like), a lubricant (e.g., talc, magnesium stearate, polyethylene glycol 6000 and the like) and the like and subjected to compression molding according to a method known per se, and where necessary, coating for taste masking, and enteric coating or sustainability according to a method known per se. Examples of the coating agent to be used therefor include hydroxypropylmethylcellulose, ethylcellulose, hydroxymethylcellulose, hydroxypropylcellulose, polyoxyethyleneglycol, Tween 80, pluronic F₆₈, cellulose acetate phthalate, hydroxypropylmethylcellulose phthalate, hydroxymethylcellulose acetate succinate; Eudragit (manufactured by Rohm, Germany, methacrylic acid/acrylic acid copolymer), dye (e.g., red iron oxide, titanium dioxide, etc.), and the like. When formulating an enteric preparation, an intermediate phase may be formed according to a method known per se for the purpose of separating an enteric phase and a drug-containing phase.

To formulate an external preparation, the compound of the present invention may be formulated into a solid, semisolid or liquid external preparation according to a method known per se. For example, to provide the above-mentioned solid preparation, the compound of the present invention may be directly used, or formulated into a powder composition by adding to mix with an excipient (e.g., glycol, mannitol, starch, microcrystalline cellulose and the like), a thickener (e.g., natural gum, cellulose derivative, acrylic acid polymer and the like) and the like. To provide the above-mentioned liquid preparation, an oily or aqueous suspension is formulated mostly in the same manner as in the injection. In the case of a semisolid preparation, an aqueous or oily gel, or an ointment form is preferable. They may contain a pH adjusting agent (e.g., carbonic acid, phosphoric acid, citric acid, hydrochloric acid, sodium hydroxide and the like), a preservative (e.g., paraoxybenzoate, chlorobutanol, benzalkonium chloride and the like) and the like. To provide a suppository, for example, the compound of the present invention can be formulated into an oily or aqueous solid, semisolid or liquid suppository according to a method known per se. Examples of the oily base to be used for the above-mentioned compositions include higher fatty acid glycerides [e.g., cacao butter, witepsol (manufactured by Dynamitnovel Ltd., Germany) and the like], intermediate grade fatty acids [e.g., miglyol (manufactured by Dynamitnovel Ltd., Germany) and the like], vegetable oil (e.g., sesame oil, soybean oil, cottonseed oil and the like) and the like. Moreover, examples of the aqueous base include polyethylene glycol and propylene glycol, and examples of the aqueous gel base include natural gums, cellulose derivatives, vinyl polymers, acrylic acid polymers and the like.

The present invention is explained in more detail in the following by referring to Formulation Examples, Reference Examples and Examples, which are not to be construed as limitative.

¹H-NMR spectra are measured with tetramethylsilane as the internal standard, using VARIAN GEMINI 200 (200 MHz type spectrometer), JEOL Ltd. (JEOL) LAMBDA300 (300 MHz type spectrometer) or BRUKER AM 500 (500 MHz type spectrometer); all δ values are expressed in ppm. Unless otherwise specified, “%” shows weight percent. However, the yield is in mol/mol %. Other symbols in the present specification mean the following.

s: singlet

d: doublet

t: triplet

dt: double triplet

m: multiplet

br: broad

The room temperature means, but is not particularly strictly limited to, the range of from about 15° C. to 25° C. In addition, lactose, cornstarch and magnesium stearate used in the Formulation Examples and Examples were the Japanese Pharmacopoeia 14th Edition or Japanese Pharmaceutical Excipients 2003 compatible products.

### EXAMPLES

### Formulation Example 1

| | | |
|---|---|---|
| (1) | compound A | 1 g |
| (2) | lactose | 197 g |
| (3) | corn starch | 50 g |
| (4) | magnesium stearate | 2 g |

The above-mentioned (1) and (2), and corn starch (20 g) were admixed, and granulated together with a paste produced from corn starch (15 g) and water (25 mL). Corn starch (15 g) and the above-mentioned (4) was added thereto, and the mixture was compressed by a tablet compression machine to give tablets (2000 tablets, diameter of 3 mm) containing 0.5 mg of compound A per tablet.

### Formulation Example 2

| | | |
|---|---|---|
| (1) | compound A | 2 g |
| (2) | lactose | 197 g |
| (3) | corn starch | 50 g |
| (4) | magnesium stearate | 2 g |

In the same manner as in Formulation Example 1, tablets (2000 tablets, diameter 3 mm) containing 1.0 mg of compound A per tablet were produced.

### Formulation Example 3

| | | |
|---|---|---|
| (1) | compound A | 5.0 mg |
| (2) | lactose | 60.0 mg |
| (3) | corn starch | 35.0 mg |
| (4) | gelatin | 3.0 mg |
| (5) | magnesium stearate | 2.0 mg |

A mixture of the above-mentioned (1), (2) and (3) was granulated by passing through a 1 mm mesh sieve while using 10% aqueous gelatin solution (0.03 ml, 3.0 mg as gelatin). The granules were dried at 40° C., and sieved again. The obtained granules were mixed with the above-mentioned (5), and compressed. The obtained core tablet was coated with an aqueous sugar coating suspension of saccharose, titanium dioxide, talc and gum arabic. The coated tablet was glazed with beeswax to give a coated tablet.

### Reference Example 1

### 2-amino-4-methyl-5-(4-nitrophenyl)thiophene-3-carboxylic acid ethyl ester

A mixture of 4-nitrophenylacetone (35.0 g, 195 mmol), ethyl cyanoacetate (23.8 g, 195 mmol), ammonium acetate (3.1 g, 40 mmol) and acetic acid (9.1 ml, 159 mmol) was heated under reflux for 24 hr while removing the resulting water by a Dean-Stark trap. After cooling, the reaction mixture was concentrated under reduced pressure, and the residue was partitioned between dichloromethane and aqueous sodium hydrogen carbonate solution. The organic layer was washed with brine and dried (MgSO₄), after which the solvent was evaporated under reduced pressure. The residue was purified by silica gel column chromatography. The obtained oily substance was dissolved in ethanol, sulfur (5.0 g, 160 mmol) and diethylamine (16.0 ml, 160 mmol) were added, and the mixture was stirred at 60-70° C. for 2 hr. After cooling, the reaction mixture was concentrated under reduced pressure, and the residue was partitioned between dichloromethane and aqueous sodium hydrogen carbonate solution. The organic layer was washed with brine and dried (MgSO₄), after which the solvent was evaporated under reduced pressure. The residue was purified by silica gel column chromatography, and crystallized from ether-hexane to give the title compound (22.2 g, 52%) as red plate crystals.

mp: 168-170° C. (recrystallization from ether-hexane).
elemental analysis value for C₁₄H₁₄N₂O₄S

| | C (%) | H (%) | N (%) |
|---|---|---|---|
| Calculated: | 54.89; | 4.61; | 9.14 |
| Found: | 54.83; | 4.90; | 9.09 |

¹H-NMR (200 MHz, CDCl₃) δ: 1.39 (3H, t, J=7.1 Hz), 2.40 (3H, s), 4.34 (2H, q, J=7.1 Hz), 6.27 (2H, br), 7.48 (2H, d, J=8.7 Hz), 8.23 (2H, d, J=8.7 Hz).

IR (KBr): 3446, 3324, 1667, 1580, 1545, 1506, 1491, 1475, 1410, 1332 cm⁻¹.

### Reference Example 2

### 5-methyl-6-(4-nitrophenyl)-3-phenylthieno[2,3-d]pyrimidine-2,4(1H,3H)-dione

To a solution of the compound (5.00 g, 16.32 mmol) obtained in Reference Example 1 in pyridine (30 ml) was added phenylisocyanate (2.66 ml, 24.48 mmol), and the mixture was stirred at 45° C. for 6 hr. The reaction mixture was concentrated under reduced pressure and the obtained residue was treated to give an ethanol (6 ml) solution. 28% Sodium methoxide (7.86 g, 40.80 mmol) was added to the solution, and the reaction mixture was stirred at room temperature for 2 hr. 2N Hydrochloric acid (25 ml, 50 mmol) was added, and the ethanol solvent was evaporated under reduced pressure. The obtained residue was filtrated and washed with water-ethanol. After drying under reduced pressure, the residue was recrystallized from ethanol to give the title compound (6.09 g, 98%) as a yellow powder.

mp: >300° C.
elemental analysis value for C₁₉H₁₃N₃O₄S•0.3H₂O

| | C (%) | H (%) | N (%) |
|---|---|---|---|
| Calculated: | 59.30; | 3.56; | 10.92 |
| Found: | 59.56; | 3.52; | 10.93 |

¹H-NMR (300 MHz, DMSO-d₆) δ: 2.50 (3H, s), 7.31-7.46 (5H, m), 7.78 (2H, d, J=8.8 Hz), 8.32 (2H, d, J=8.8 Hz), 12.50 (1H, s).

IR (KBr): 1715, 1657, 1593, 1510 cm⁻¹.

### Reference Example 3

### 1-(2,6-difluorobenzyl)-5-methyl-6-(4-nitrophenyl)-3-phenylthieno[2,3-d]pyrimidine-2,4(1H,3H)-dione

To a solution of the compound (52.54 g, 0.131 mol) obtained in Reference Example 2 in dimethylformamide (1.0 l) were added potassium carbonate (19.00 g, 0.138 mol), potassium iodide (22.90 g, 0.138 mol) and 2,6-difluorobenzylchloride (22.40 g, 0.138 mol), and the mixture was stirred at room temperature for 2 hr. The reaction mixture was concentrated, and the obtained residue was partitioned between chloroform and brine. The aqueous layer was extracted with chloroform. The extracts were combined and washed with brine and dried (MgSO₄), after which the solvent was evaporated under reduced pressure. The obtained residue was purified by silica gel column chromatography to give the title compound (61.50 g, 93%) as pale-yellow crystals.

mp: 280-282° C.
elemental analysis value for C₂₆H₁₇N₃O₄SF₂

| | C (%) | H (%) | N (%) |
|---|---|---|---|
| Calculated: | 61.78; | 3.39; | 8.31 |
| Found: | 61.67; | 3.46; | 8.21 |

¹H-NMR (300 MHz, CDCl₃) δ: 2.57 (3H, s), 5.38 (2H, s), 6.94 (2H, d, J=8.1 Hz), 7.42-7.58 (8H, m), 8.29 (2H, d, J=8.8 Hz).

IR (KBr): 1719, 1669, 1524, 1473 cm⁻¹.

### Reference Example 4

### 5-bromomethyl-1-(2,6-difluorobenzyl)-6-(4-nitrophenyl)-3-phenylthieno[2,3-d]pyrimidine-2,4(1H,3H)-dione

A mixture of the compound (30.34 g, 0.060 mol) obtained in Reference Example 3, N-bromosuccinimide (12.81 g, 0.072 mol), α,α′-azobisisobutyronitrile (1.15 g, 0.007 mol) and chlorobenzene (450 ml) was stirred at 85° C. for 3 hr. After cooling, the reaction mixture was washed with brine and dried (MgSO₄), after which the solvent was evaporated under reduced pressure. The obtained residue was recrystallized from ethyl acetate to give the title compound (80.21 g, 100%) as yellow needle-like crystals.

mp: 228-229° C.

¹H-NMR (300 MHz, CDCl₃) δ: 4.77 (2H, s), 5.38 (2H, s), 6.96 (2H, t, J=8.1 Hz), 7.29-7.58 (6H, m), 7.79 (2H, d, J=8.5 Hz), 8.35 (2H, d, J=8.5 Hz).

IR (KBr): 1721, 1680, 1524, 1473, 1348 cm⁻¹.

FAB-Mass m/z 584 (MH)⁺

### Reference Example 5

### 5-(N-benzyl-N-methylaminomethyl)-1-(2,6-difluorobenzyl)-6-(4-nitrophenyl)-3-phenylthieno[2,3-d]pyrimidine-2,4(1H,3H)-dione

To a solution of the compound (80.00 g, 0.119 mol) obtained in Reference Example 4 in dimethylformamide (600 ml) were added ethyldiisopropylamine (27.00 ml, 0.155 mol) and benzylmethylamine (18.45 ml, 0.143 mol) under ice-cooling. The mixture was stirred at room temperature for 2 hr. The reaction mixture was concentrated, and the obtained residue was partitioned between ethyl acetate and saturated aqueous sodium hydrogen carbonate solution. The aqueous layer was extracted with ethyl acetate, and the organic layers were combined and dried (MgSO₄), after which the solvent was evaporated under reduced pressure. The obtained residue was purified by silica gel column chromatography to give a yellow oil (74.90 g, 100%), which was recrystallized from ethyl acetate to give the title compound as yellow needle-like crystals.

mp: 173-174° C.
elemental analysis value for C₃₄H₂₆N₄O₄SF₂•0.5H₂O

| | C (%) | H (%) | N (%) |
|---|---|---|---|
| Calculated: | 64.45; | 4.29; | 8.84 |
| Found: | 64.50; | 4.24; | 8.82 |

¹H-NMR (300 MHz, CDCl₃) [free amine] δ: 1.31 (3H, s), 3.60 (2H, s), 3.96 (2H, s), 5.39 (2H, s), 6.95 (2H, t, J=8.2 Hz), 7.18-7.55 (11H, m), 8.02 (2H, d, J=9.0 Hz), 8.26 (2H, d, J=9.0 Hz).

IR (KBr) [hydrochloride]: 1719, 1678, 1597, 1520 cm⁻¹.

### Reference Example 6

### 6-(4-aminophenyl)-5-(N-benzyl-N-methylaminomethyl)-1-(2,6-difluorobenzyl)-3-phenylthieno[2,3-d]pyrimidine-2,4(1H,3H)-dione

To a solution of the compound (3.00 g, 4.80 mmol) obtained in Reference Example 5 in formic acid (30 ml) were added 1M hydrogen chloride-ether (14.4 ml, 14.4 mmol) and 10% palladium carbon powder (300 mg) under ice-cooling. The mixture was stirred for 2 hr at ambient temperature under normal pressure, and hydrogenated. The reaction mixture was filtered through celite. The filtrate was concentrated under reduced pressure, and the obtained residue was partitioned between dichloromethane and saturated aqueous sodium hydrogen carbonate solution. The aqueous layer was extracted with dichloromethane, the organic layers were combined and dried (MgSO₄), after which the solvent was evaporated under reduced pressure. The obtained residue was purified by silica gel column chromatography to give the title compound (2.41 g, 84%) as white crystals.

| mp: 205-207° C. | | | |
|---|---|---|---|
| elemental analysis value for C₃₄H₂₈N₄O₂SF₂•0.1AcOEt•1.2H₂O | | | |
| | C (%) | H (%) | N (%) |
| Calculated: | 66.09; | 5.03; | 8.96 |
| Found: | 66.93; | 4.94; | 8.67 |

¹H-NMR (300 MHz, CDCl₃) δ: 2.05 (3H, s), 3.56 (2H, s), 3.83 (2H, br), 3.88 (2H, s), 5.36 (2H, s), 6.70 (2H, d, J=8.8 Hz), 6.88-6.94 (2H, m), 7.21-7.31 (8H, m), 7.41-7.53 (5H, m).

IR (KBr): 1715, 1657, 1628, 1537 cm⁻¹.

### Reference Example 7

### 5-(N-benzyl-N-methylaminomethyl)-1-(2,6-difluorobenzyl)-6-[4-(3-methoxyureido)phenyl]-3-phenylthieno[2,3-d]pyrimidine-2,4(1H,3H)-dione

### Compound A

To a solution of the compound (5.0 g, 8.41 mmol) obtained in Reference Example 6 in dichloromethane (120 ml) was added triethylamine (2.34 ml, 16.82 mmol) under ice-cooling, and the mixture was stirred. Under ice-cooling, N,N′-carbonyldiimidazole (2.73 g, 16.82 mmol) was added to the reaction mixture. The temperature of the mixture was raised from ice-cooling to room temperature and stirred for 42 hr. The mixture was placed under ice-cooling again, and O-methylhydroxylamine hydrochloride (7.02 g, 84.08 mmol) and triethylamine (11.7 ml, 84.08 mmol) were added. The reaction mixture was raised from ice-cooling to room temperature and stirred for 3 hr. The reaction mixture was partitioned between chloroform and saturated aqueous sodium hydrogen carbonate solution. The aqueous layer was extracted with chloroform. The extracts were combined and washed with brine and dried (MgSO₄), after which the solvent was evaporated under reduced pressure. The obtained residue was purified by silica gel column chromatography to give a pale-yellow solid, which was recrystallized from chloroform-ether to give the title compound (4.52 g, 80%) as white crystals.

mp: 204-205° C.
elemental analysis value for C₃₆H₃₁N₅O₄SF₂

| | C (%) | H (%) | N (%) |
|---|---|---|---|
| Calculated: | 64.75; | 4.68; | 10.49 |
| Found: | 64.61; | 4.67; | 10.31 |

¹H-NMR (300 MHz, CDCl₃) δ: 2.05 (3H, s), 3.57 (2H, s), 3.82 (3H, s), 3.90 (2H, s), 5.37 (2H, s), 6.92 (2H, d, J=8.2 Hz), 7.16-7.31 (9H, m), 7.42-7.57 (5H, m), 7.63 (1H, s), 7.73 (2H, d, J=8.8 Hz).

IR (KBr): 3338, 3064, 1717, 1669, 1628, 1591, 1531, 1470 cm⁻¹.

### Reference Example 8

### 5-(N-benzyl-N-methylaminomethyl)-1-(2,6-difluorobenzyl)-6-[4-(3-methoxyureido)phenyl]-3-phenylthieno[2,3-d]pyrimidine-2,4(1H,3H)-dione hydrochloride

To a solution of the white crystals (38.34 g, 57.42 mmol) obtained in Reference Example 7 in dichloromethane (800 ml) was added 1M hydrogen chloride in ether (100 ml) under ice-cooling, and the mixture was stirred at the same temperature for 10 min. The reaction mixture was concentrated under reduced pressure, and the obtained residue was recrystallized from methanol-ether to give the title compound (40.0 g, 99%) as white powder crystals.

mp: 182-185° C.
elemental analysis value for C₃₆H₃₁N₅O₄SF₂•HC1•0.5H₂O

| | C (%) | H (%) | N (%) |
|---|---|---|---|
| Calculated: | 60.63; | 4.66; | 9.82 |
| Found: | 60.45; | 4.68; | 9.62 |

| | |
|---|---|
| IR (KBr): | 3440, 3042, 1713, 1665, 1628, 1593, 1539, 1473 cm⁻¹. |

FAB-Mass m/z 668 (MH)⁺

### Example 1

Using the compound (100 mg) produced in Reference Example 7, lactose (165 mg), corn starch (25 mg), polyvinyl alcohol (4 mg) and magnesium stearate (1 mg), a tablet is produced according to a conventional method.

### Example 2

Using the compound (100 mg) produced in Reference Example 8, lactose (165 mg), corn starch (25 mg), polyvinyl alcohol (4 mg) and magnesium stearate (1 mg), a tablet is produced according to a conventional method.

### Example 3

Using the compound (1 g) produced in Reference Example 7, lactose (197 g), corn starch (50 g) and magnesium stearate (2 g), a tablet is produced according to a conventional method.

### Example 4

Using the compound (1 g) produced in Reference Example 8, lactose (197 g), corn starch (50 g) and magnesium stearate (2 g), a tablet is produced according to a conventional method.

### Reference Example 9

### Production of 2-[N-(2,6-difluorobenzyl)-N-ethoxycarbonyl]amino-4-[N-(2-methoxyethyl)-N-methylaminomethyl]-5-(4-aminophenyl)thiophene-3-carboxylic acid ethyl ester

To a solution of 2-[N-(2,6-difluorobenzyl)-N-ethoxycarbonyl]amino-4-[N-(2-methoxyethyl)-N-methylaminomethyl]-5-(4-nitrophenyl)thiophene-3-carboxylic acid ethyl ester (12.43 g) (JP-A-2001-278884, WO00/56739) in ethanol (315 ml) were added 2N-hydrogen chloride/diethyl ether solution (21 ml) and 10% palladium/carbon containing 50% water (3.73 g), and the mixture was vigorously stirred for 1 hr under a hydrogen atmosphere. The filtrate free of the catalyst was neutralized with aqueous sodium hydrogen carbonate solution, after which the solvent was evaporated. The obtained residue was partitioned between ethyl acetate/water. The organic layer was washed with saturated brine, and dried over anhydrous magnesium sulfate. The solvent was evaporated under reduced pressure, and the obtained residue was purified by NH-silica gel (manufactured by Fuji Silysia Chemical Ltd.) chromatography to give the title compound (11.44 g) as an oil.

¹H-NMR (CDCl₃) δ: 1.12-1.30 (3H, br), 2.05 (3H, s), 2.39 (2H, t, J=6.3 Hz), 3.27 (3H, s), 3.32 (3H, t, J=6.3 Hz), 3.59 (2H, s), 3.78 (2H, s), 4.20 (2H, q, J=7.1 Hz), 4.10-4.23 (2H, br), 5.00 (2H, s), 6.66 (2H, d, J=8.6 Hz), 6.84 (2H, t, J=8.2 Hz), 7.18 (2H, d, J=8.6 Hz), 7.15-7.30 (1H, m).

IR (KBr): 1717, 1626, 1609, 1472, 1406, 1300, 1246 cm⁻¹.

### Reference Example 10

### Production of 2-[(2,6-difluorobenzyl)(ethoxycarbonyl)amino]-5-(4-{[(methoxyamino)carbonyl]amino}phenyl)-4-{[(2-methoxyethyl)(methyl)amino]methyl}-3-thiophenecarboxylic acid ethyl ester

To a solution (113 ml) of the compound (4.89 g) of Reference Example 9 in dichloromethane was added N-ethyldiisopropylamine (3.06 ml) under ice-cooling, and the mixture was stirred. N,N′-Carbonyldiimidazole (2.82 g) was added to the reaction mixture under ice-cooling. The temperature of the mixture was raised from ice-cooling to room temperature, and the mixture was stirred for 67 hr. The mixture was placed under ice-cooling again, and O-methylhydroxyamine hydrochloride (7.26 g) and N-ethyldiisopropylamine (15.6 ml) were added. The temperature of the reaction mixture was raised from ice-cooling to room temperature, and the mixture was stirred for 19 hr. The mixture was partitioned between chloroform and saturated aqueous sodium hydrogen carbonate solution. The aqueous layer was extracted with chloroform, and the extracts were combined and washed with brine and dried (MgSO₄), after which the solvent was evaporated under reduced pressure. The obtained residue was purified by silica gel column chromatography to give the title compound (4.89 g) in a pale-yellow caramel form.

¹H-NMR (CDCl₃) δ: 1.19 (3H, brs), 1.30 (3H, t, J=6.9 Hz), 2.04 (3H, s), 2.40 (2H, t, J=6.0 Hz), 3.27 (3H, s), 3.33 (2H, t, J=6.0 Hz), 3.60 (2H, s), 3.81 (3H, s), 4.13-4.24 (4H, m), 5.00 (2H, s), 6.84 (2H, t, J=7.8 Hz), 7.19-7.29 (2H, m), 7.36 (2H, d, J=8.7 Hz), 7.50 (2H, d, J=8.7 Hz), 7.60 (1H, s).

IR (KBr): 1717, 1590, 1528, 1472, 1408, 1304 cm⁻¹.

### Reference Example 11

### Production of 2-[(2,6-difluorobenzyl)(ethoxycarbonyl)amino]-5-(4-{[(methoxyamino)carbonyl]amino}phenyl)-4-{[(2-methoxyethyl)(methyl)amino]methyl}-3-thiophenecarboxylic acid

To a solution of the compound (4.81 g) of Reference Example 10 in ethanol (114 ml) was added 2N sodium hydroxide solution (18.9 ml), and the mixture was stirred at 60° C. for 5 hr. The mixture was cooled to room temperature, 1N hydrochloric acid (37.8 ml) was added, and the solvent was evaporated. The obtained residue was dissolved in ethanol and toluene, and the solvent was evaporated again. Anhydrous ethanol (30 ml) was added to the residue, and the inorganic product was filtrated. The filtrate was concentrated to dryness, and the obtained residue was triturated with anhydrous ether, collected by filtration and dried to give the title compound (4.43 g).

¹H-NMR (CDCl₃) δ: 1.17 (3H, brs), 2.45 (3H, s), 2.81 (2H, brs), 3.28 (3H, s), 3.55 (2H, t, J=4.8 Hz), 3.82 (3H, s), 3.92 (2H, s), 4.10-4.35 (2H, m), 5.06 (2H, s), 6.82 (2H, t, J=7.8 Hz), 7.16 (2H, d, J=8.4 Hz), 7.22-7.35 (1H, m), 7.60 (2H, d, J=8.4 Hz), 8.00-8.50 (2H, br). IR (KBr): 1713, 1605, 1528, 1472, 1408 cm⁻¹.

### Reference Example 12

### Production of N-(4-(1-(2,6-difluorobenzyl)-5-(((2-methoxyethyl)(methyl)amino)methyl)-2,4-dioxo-3-(2-pyridinyl)-1,2,3,4-tetrahydrothieno[2,3-d]pyrimidin-6-yl)phenyl)-N′-methoxyurea

To a solution of the compound (607 mg) obtained in Reference Example 11 and 2-aminopyridine (142 mg) in DMF (10 ml) were added diethyl cyanophosphate (245 mg) and N-ethyldiisopropylamine (284 μl). The mixture was gradually warmed to room temperature and stirred for 13 hr, after which the reaction mixture was partitioned between ethyl acetate/water. The organic layer was washed successively with water and saturated brine and dried over anhydrous sodium sulfate. The residue obtained by evaporating the solvent under reduced pressure was crudely purified by aminopropylsilica gel (manufactured by Fuji Silysia Chemical Ltd.) chromatography. The obtained crude amide form (350 mg) was dissolved in ethanol (25.5 ml), a solution (196 mg) of 28%-sodium methoxide in methanol was added, and the mixture was stirred at room temperature for 15 hr. The mixture was neutralized with 1N-hydrochloric acid (1 ml), the solvent was evaporated, and the residue was partitioned between ethyl acetate/water. The organic layer was washed successively with water and saturated brine and dried over anhydrous sodium sulfate. The residue obtained by evaporating the solvent under reduced pressure was purified by aminopropylsilica gel (manufactured by Fuji Silysia Chemical Ltd.) chromatography (45 g; developer; ethyl acetate/hexane:7/3→ethyl acetate) and recrystallized from THF-ethanol to give the title compound (210 mg) as colorless crystals.

elemental analysis for C₃₁H₃₀N₆O₅SF₂

Calculated: C, 58.48; H, 4.75; N, 13.20. Found: C, 58.46; H, 4.68; N, 12.93.

¹H-NMR (CDCl₃) δ: 2.15 (3H, s), 2.62 (2H, t, J=5.9 Hz), 3.26 (3H, s), 3.41 (2H, t, J=5.9 Hz), 3.80 (3H, s), 3.81 (2H, brs), 5.34 (2H, brs), 6.91 (2H, t, J=8.1 Hz), 7.24-7.40 (4H, m), 7.53 (2H, d, J=8.4 Hz), 7.62 (2H, d, J=8.4 Hz), 7.65 (1H, s), 7.88 (1H, dt, J=1.5 Hz, 7.8 Hz), 8.67-8.69 (1H, m).

IR (KBr): 1717, 1674, 1591, 1530, 1460, 1329 cm⁻¹.

### Reference Example 13

### Production of N-(4-(5-((benzyl(methyl)amino)methyl)-1-(2,6-difluorobenzyl)-2,4-dioxo-3-(2-pyridinyl)-1,2,3,4-tetrahydrothieno[2,3-d]pyrimidin-6-yl)phenyl)-N′-methoxyurea

To a solution (20 ml) of 4-(N-benzyl-N-methylaminomethyl)-2-[N-(2,6-difluorobenzyl)-N-ethoxycarbonyl]amino-5-[4-(3-methoxyureido)phenyl]thiophene-3-carboxylic acid (2.40 g, 3.76 mmol) and 2-aminopyridine (1.06 g, 11.28 mmol) in DMF were added ethyldiisopropylamine (1.05 ml, 6.02 mmol) and diethyl cyanophosphorate (0.86 ml, 5.64 mmol), and the mixture was stirred at room temperature for 3 days. An aqueous sodium hydrogen carbonate solution was added, the mixture was extracted with ethyl acetate, and the organic layer was washed with brine. After drying over magnesium sulfate, the mixture was concentrated under reduced pressure, and the residue was purified by silica gel column chromatography (eluate; ethyl acetate) to give an amide. The obtained amide was dissolved in methanol (40 ml), and sodium methoxide (2.03 mg, 37.6 mmol) was added. After stirring at room temperature for 5 hr, the mixture was concentrated, neutralized with 1N hydrochloric acid, and extracted with ethyl acetate. The organic layer was washed with brine, dried over magnesium sulfate, and concentrated under reduced pressure. The residue was purified by NH-silica gel (manufactured by Fuji Silysia Chemical Ltd.) column chromatography (eluate; ethyl acetate) to give the title compound (1.59 g, 63%) in a pale-yellow amorphous form.

¹H-NMR (CDCl₃) δ: 2.05 (3H, s), 3.56 (2H, s), 3.82 (3H, s), 3.89 (2H, s), 5.34 (2H, brs), 6.91 (2H, t, J=8.0 Hz), 7.1-7.45 (9H, m), 7.56 (2H, d, J=8.8 Hz), 7.65 (1H, s), 7.75 (2H, d, J=8.8 Hz), 7.91 (1H, dt, J=2.0, 7.7 Hz), 8.7-8.75 (1H, m).

elemental analysis for C₃₅H₃₀F₂N₆O₄S₂

Calculated: C, 62.86; H, 4.52; N, 12.57. Found: C, 62.72; H, 4.31; N, 12.40.

mp 179-182° C.

### Reference Example 14

### Production, of N-(4-(1-(2,6-difluorobenzyl)-5-((methylamino)methyl)-2,4-dioxo-3-(2-pyridinyl)-1,2,3,4-tetrahydrothieno[2,3-d]pyrimidin-6-yl)phenyl)-N′-methoxyurea

To a solution of the compound (1.59 g, 2.38 mmol) of Reference Example 13 in ethanol (40 ml) were added 1N hydrochloric acid (7 ml) and 10% palladium/carbon containing 50% water (0.63 g), and the mixture was vigorously stirred for 20 hr under a hydrogen atmosphere. The filtrate free of the catalyst was neutralized with 1N aqueous sodium hydroxide solution, after which the solvent was evaporated. The obtained residue was partitioned between ethyl acetate/water, and the organic layer was washed with saturated brine, and dried over anhydrous magnesium sulfate. Under reduced pressure, the solvent was evaporated, and the obtained powder was washed with diethyl ether to give the title compound (980 mg, 71%) as a pale-yellow powder.

¹H-NMR (CDCl₃) δ: 2.34 (3H, s), 3.78 (2H, s), 3.82 (2H, s), 5.38 (2H, brs), 6.92 (2H, t, J=8.2 Hz), 7.2-7.8 (9H, m), 7.92 (1H, dt, J=1.8 Hz, 7.6 Hz), 8.72 (1H, d, J=4.8 Hz).

### Reference Example 15

### Production of N-(4-(1-(2,6-difluorobenzyl)-5-(((2-ethoxyethyl)(methyl)amino)methyl)-2,4-dioxo-3-(2-pyridinyl)-1,2,3,4-tetrahydrothieno[2,3-d]pyrimidin-6-yl)phenyl)-N′-methoxyurea

To a solution (4.3 ml) of the compound (251 mg) of Reference Example 14 in DMF were added 2-ethoxyethylchloride (141 mg), N-ethyldiisopropylamine (245 μl) and potassium iodide (107 mg), and the mixture was stirred at 60° C. for 24 hr. The reaction mixture was partitioned between ethyl acetate/water, and the organic layer was washed with saturated brine, and dried over anhydrous sodium sulfate. The residue obtained by evaporating the solvent was purified by aminopropylsilica gel (manufactured by Fuji Silysia Chemical Ltd.) chromatography (45 g; developer; ethyl acetate/hexane:3/2→4/1), and recrystallized from ethyl acetate to give the title compound (62 mg) as colorless crystals.

elemental analysis for C₃₂H₃₂N₆O₅SF₂.0.1AcOEt

Calculated: C, 59.01; H, 5.01; N, 12.74. Found: C, 59.11; H, 5.13; N, 12.55.

¹H-NMR (CDCl₃) δ: 1.13 (3H, t, J=6.9 Hz), 2.15 (3H, s), 2.63 (2H, t, J=6.2 Hz), 3.39 (2H, q, J=6.9 Hz), 3.44 (2H, t, J=6.2 Hz), 3.80 (2H, brs), 3.81 (3H, s), 5.34 (2H, brs), 6.91 (2H, t, J=8.1 Hz), 7.19 (1H, s), 7.27-7.32 (1H, m), 7.35-7.41 (2H, m), 7.53 (2H, d, J=8.4 Hz), 7.63 (1H, s), 7.64 (2H, d, J=8.4 Hz), 7.88 (1H, dt, J=1.2 Hz, 7.5 Hz), 8.68 (1H, dt, J=0.9 Hz, 4.8 Hz). IR (KBr): 1717, 1674, 1591, 1530, 1460, 1329 cm⁻¹.

### Example 5

Using the compound (100 mg) produced in Reference Example 12, lactose (165 mg), corn starch (25 mg), polyvinyl alcohol (4 mg) and magnesium stearate (1 mg), a tablet is produced according to a conventional method.

### Example 6

Using the compound (100 mg) produced in Reference Example 15, lactose (165 mg), corn starch (25 mg), polyvinyl alcohol (4 mg) and magnesium stearate (1 mg), a tablet is produced according to a conventional method.

### Example 7

Using the compound (1 g) produced in Reference Example 12, lactose (197 g), corn starch (50 g) and magnesium stearate (2 g), a tablet is produced according to a conventional method.

### Example 8

Using the compound (1 g) produced in Reference Example 15, lactose (197 g), corn starch (50 g) and magnesium stearate (2 g), a tablet is produced according to a conventional method.

### INDUSTRIAL APPLICABILITY

The premature ovulation inhibitor for use in in vitro fertilization or embryo transfer process of the present invention, which comprises a nonpeptidic compound having a gonadotropin releasing hormone antagonistic action, is low toxic, permits oral administration, and has a superior inhibitory effect on premature ovulation in in vitro fertilization or embryo transfer process. That is, the premature ovulation inhibitor for use in in vitro fertilization or embryo transfer process of the present invention, which comprises a nonpeptidic compound having a gonadotropin releasing hormone antagonistic action, has a superior GnRH antagonistic action, is low toxic, and is superior in oral absorbability, action sustainability, stability and pharmacokinetics. Thus, it can be safely used for promoting or assisting in vitro fertilization or embryo transfer process. In addition, production of the premature ovulation inhibitor of the present invention is easy and simple.

This application is based on a patent application No. 2005-212973 filed in Japan, the contents of which are incorporated in full herein by this reference.

1. A premature ovulation inhibitor for use in in vitro fertilization or embryo transfer process, comprising a nonpeptidic compound having a gonadotropin releasing hormone antagonistic action.

2. The inhibitor of claim 1, wherein the compound is represented by the formula 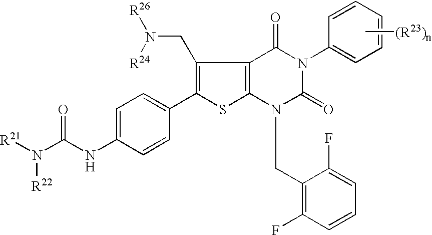
wherein R²¹ and R²² are each (1) a hydrogen atom, (2) hydroxy, (3) C₁₋₄ alkoxy, (4) C₁₋₄ alkoxy-carbonyl or (5) C₁₋₄ alkyl optionally having substituent(s),
R²³ is (1) a hydrogen atom, (2) a halogen atom, (3) hydroxy or (4) C₁₋₄ alkoxy optionally having substituent(s), or
the adjacent two R²³ are optionally bonded to form C₁₋₄ alkylenedioxy,
R²⁴ is (1) a hydrogen atom or (2) C₁₋₄ alkyl, and
R²⁶ is (1) C₁₋₄ alkyl optionally having substituent(s) or (2) a group represented by the formula 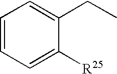
wherein R²⁵ is a hydrogen atom, or optionally bonded to R²⁴ to form heterocycle, and n is an integer of 0 to 5,
or a salt thereof or a prodrug thereof.

3. The inhibitor of claim 1, wherein the compound is represented by the formula 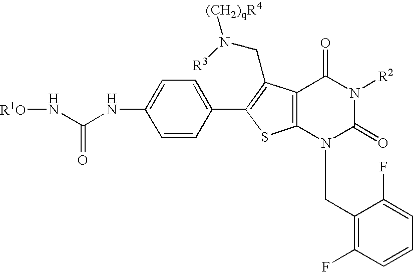
wherein
R¹ is C₁₋₄ alkyl,
R² is
(1) C₁₋₆ alkyl optionally having substituent(s) selected from (1′) a hydroxyl group, (2′) C₁₋₄ alkoxy, (3′) C₁₋₄ alkoxy-carbonyl, (4′) di-C₁₋₄ alkyl-carbamoyl, (5′) a 5- to 7-membered nitrogen-containing heterocyclic group, (6′) C₁₋₄ alkyl-carbonyl and (7′) halogen,
(2) C₃₋₈ cycloalkyl optionally having (1′) a hydroxyl group or (2′) mono-C₁₋₄ alkyl-carbonylamino,
(3) a 5- to 7-membered nitrogen-containing heterocyclic group optionally having substituent(s) selected from (1′) halogen, (2′) a hydroxyl group, (3′) C₁₋₄ alkyl and (4′) C₁₋₄ alkoxy,
(4) phenyl optionally having substituent(s) selected from (1′) halogen, (2′) C₁₋₄ alkoxy-C₁₋₄ alkyl, (3′) mono-C₁₋₄ alkyl-carbamoyl-C₁₋₄ alkyl, (4′) C₁₋₄ alkoxy and (5′) mono-C₁₋₄ alkylcarbamoyl-C₁₋₄ alkoxy or
(5) C₁₋₄ alkoxy,
R³ is C₁₋₄ alkyl,
R⁴ is (1) a hydrogen atom, (2) C₁₋₄ alkoxy, (3) C₆₋₁₀ aryl, (4) N—C₁₋₄ alkyl-N—C₁₋₄ alkylsulfonylamino, (5) a hydroxyl group or (6) a 5- to 7-membered nitrogen-containing heterocyclic group optionally having substituent(s) selected from (1′) oxo, (2′) C₁₋₄ alkyl, (3′) hydroxy-C₁₋₄ alkyl, (4′) C₁₋₄ alkoxy-carbonyl, (5′) mono-C₁₋₄ alkyl-carbamoyl and (6′) C₁₋₄ alkylsulfonyl,
q is an integer of 1 to 4,
(provided that when R² is phenyl optionally having substituent(s), R⁴ should be a 5- to 7-membered nitrogen-containing heterocyclic group optionally having substituent(s) selected from (1) oxo, (2) hydroxy-C₁₋₄ alkyl, (3) C₁₋₄ alkoxy-carbonyl, (4) mono-C₁₋₄ alkyl-carbamoyl and (5) C₁₋₄ alkylsulfonyl) or a salt thereof or a prodrug thereof.

4. The inhibitor of claim 1, which is an oral preparation.

5. A method of inhibiting premature ovulation in in vitro fertilization or embryo transfer process, which comprises administering an effective amount of the compound of claim 1 to a mammal.

6. Use of the compound of claim 1 for the production of a premature ovulation inhibitor for in vitro fertilization or embryo transfer process.

7. The inhibitor of claim 2, which is an oral preparation.

8. The inhibitor of claim 3, which is an oral preparation.
